(19) United States
(12) Patent Application Publication
(10) Pub. No.: US 2026/0235603 A1
(43) Pub. Date: Aug. 13, 2026

(54) HEMAGGLUTINATION INHIBITION ASSAYS FOR THE EVALUATION OF SEASONAL INFLUENZA VACCINES

(71) Applicant: NOVAVAX, INC., Gaithersburg, MD (US)

(72) Inventors: Raj KALKERI, Gaithersburg, MD (US); Mingzhu ZHU, Gaithersburg, MD (US); Joyce S. PLESTED, Gaithersburg, MD (US); Timothy VINCENT, Gaithersburg, MD (US); Wendy Shane CLONEY-CLARK, Gaithersburg, MD (US)

(21) Appl. No.: 19/533,775

(22) Filed: Feb. 9, 2026

Related U.S. Application Data
(60) Provisional application No. 63/755,810, filed on Feb. 7, 2025.

(51) Int. Cl. G01N 33/569

(52) U.S. Cl. CPC G01N 33/56983; G01N 2333/11; G01N 2469/20; G01N 2500/04

(57) ABSTRACT

Disclosed are methods for detecting if a biological sample (e.g., saliva, serum, blood, plasma) contains antibodies that inhibit influenza virus hemagglutinin (HA) glycoprotein hemagglutination activity. The method includes reducing or removing non-specific inhibitors of HA from the biological sample to produce a treated sample; exposing the treated sample to an influenza virus having an HA glycoprotein to produce a sample-virus mixture; incubating the sample-virus mixture with human red blood cells (RBCs), the human RBCs being at a concentration of about 0.5% to about 1%; and detecting agglutination of the human RBCs. Assay kits and methods for using the assay to select an immunogenic composition for use as a vaccine are also provided.

### RELATED APPLICATION

The present application claims priority to U.S. Provisional Patent Application No. 63/755,810, filed on Feb. 7, 2025, the contents of which is incorporated by reference in its entirety.

### FIELD OF THE INVENTION

The present disclosure is generally related to methods for evaluating efficacy of a seasonal influenza vaccine.

### BACKGROUND OF THE INVENTION

Seasonal influenza virus infections pose a significant threat to public health globally, resulting in 3-5 million cases of severe illness and 290,000-650,000 deaths, annually. Annual vaccination is recommended as the most effective approach for the prevention and control of seasonal influenza. However, currently available seasonal influenza vaccines confer variable protection due to antigenic changes resulting from the accumulation of diverse mutations.

The viral hemagglutinin (HA) surface glycoproteins are key determinants of vaccine efficacy against seasonal circulating strains of influenza. The traditional hemagglutination inhibition (HAI) assay is considered the gold standard method for determining an influenza vaccine-elicited immune response and quantitative antibody titers for the influenza virus. However, new influenza vaccine development is challenging due, in part, to traditional, labor-intensive, and cumbersome HAI assay methods involving avian red blood cells (RBCs), complex sample preparation, subjectivity, and low assay throughput. In addition, certain clades of A/H3N2 viruses agglutinate avian RBCs poorly and alternative assays, such as virus neutralization must be used.

Thus, there is a need for novel assay protocols that generate more reliable and consistent data for evaluating the efficacy of new seasonal influenza vaccines.

### SUMMARY OF THE INVENTION

The present disclosure provides methods for evaluating anti-influenza HA titer using human RBCs.

In Aspect 1, methods for determining if a biological sample contains antibodies that inhibit influenza virus hemagglutinin (HA) glycoprotein hemagglutination activity, are provided. The methods include (a) reducing or removing non-specific inhibitors of HA from the biological sample to produce a treated sample; (b) exposing the treated sample to an influenza virus having an HA glycoprotein to produce a sample-virus mixture; (c) incubating the sample-virus mixture with human red blood cells (RBCs), the human RBCs being at a concentration of about 0.5% to about 1%; and (d) detecting agglutination of the human RBCs; where the biological sample contains antibodies that inhibit HA glycoprotein hemagglutination activity if agglutination of the RBCs is not detected.

In Aspect 2, the method of Aspect 1 includes the use of a biological sample that is serially diluted, and each serial dilution of the sample is subjected to steps (a), (b), (c), and (d).

In Aspect 3, the biological sample of Aspect 2 is serially diluted 1:2.

In Aspect 4, the method of Aspect 2 or Aspect 3 can include a step of determining the hemagglutination inhibition titer of the biological sample.

In Aspect 5, the method of any one of Aspects 1-4 includes the use of human RBCs that are at a concentration of about 0.75%.

In Aspect 6, the sample-virus mixture of any one of Aspects 1-5 is incubated with the human RBCs for about 60 minutes to about 150 minutes, or about 80 minutes to about 100 minutes.

In Aspect 7, step (a) of any one of Aspects 1-6 is performed using a receptor-destroying enzyme (RDE).

In Aspect 8, the RDE of Aspect 7 is heat-inactivated prior to step (b).

In Aspect 9, the sample-virus mixture of any one of Aspects 1-8 includes 2 to 10 hemagglutination (HAg) units, or 3 to 5 HAg units, or 4 HAg units of virus.

In Aspect 10, the virus of Aspect 9 comprises an egg-derived influenza virus.

In Aspect 11, the virus of Aspect 9 comprises a virus-like particle (VLP).

In Aspect 12, the influenza virus used in any one of Aspects 1-11 is treated with oseltamivir.

In Aspect 13, the VLP of Aspect 11 lacks a functional NA glycoprotein.

In Aspect 14, step (d) of any one of Aspects 1-13 is performed using an automated image reader-based analysis of hemagglutination.

In Aspect 15, the method of any one of Aspects 1-14 is performed once per biological sample.

In Aspect 16, a kit is provided for use in a method of any one of Aspects 1-15. The kit comprises an influenza virus having an HA glycoprotein; and human RBCs and/or an RDE.

In Aspect 17, the influenza virus of Aspect 16 comprises an egg-derived influenza virus.

In Aspect 18, the kit of Aspect 17 comprises oseltamivir or the egg-derived influenza virus has been treated with oseltamivir.

In Aspect 19, the influenza virus of Aspect 16 comprises a VLP.

In Aspect 20, the kit of Aspect 19 comprises oseltamivir, the VLP has been treated with oseltamivir, or the VLP lacks a functional NA glycoprotein.

In Aspect 21, the kit of any one of Aspects 16-20 comprises at least one of a dilution buffer; a multi-well assay plate; and an RDE.

In Aspect 22, the dilution buffer of Aspect 21 comprises Dulbecco's phosphate buffered saline.

In Aspect 23, a method for selecting an immunogenic composition for use as a vaccine is provided. The method includes (a) obtaining a plurality of first biological samples from subjects that have been administered a first immunogenic composition directed to a seasonal influenza virus; (b) performing a method according to claim 4 using the plurality of first biological samples to determine the hemagglutination inhibition titers of the first biological samples; and (c) selecting the first immunogenic composition for use as a vaccine if the hemagglutination inhibition titers in the plurality of first biological samples meets or exceeds a predetermined threshold.

In Aspect 24, the method of Aspect 23 also includes (d) obtaining a plurality of second biological samples from subjects that have been administered a second immunogenic composition directed to a seasonal influenza virus; (e) performing a method according to claim 4 using the plurality of second biological samples to determine the hemagglutination inhibition titers of the second biological samples; (f) comparing the hemagglutination inhibition titers of the plurality of first biological samples to the hemagglutination inhibition titers of the second biological samples; and (g) selecting the immunogenic composition that was administered to the subjects with biological samples having higher hemagglutination inhibition titers for use as a vaccine.

### BRIEF DESCRIPTION OF THE FIGURES

The invention may be more completely understood in consideration of the following detailed description of various embodiments of the invention in connection with the accompanying drawings, in which:

FIG. 1A shows a linear relationship between expected geometric mean titer (GMT) and GMT observed using an egg-derived virus HAI assay described herein for influenza virus strain A/Kansas/14/2017. R² is coefficient of determination.

FIG. 1B shows a linear relationship between expected geometric mean titer (GMT) and GMT observed using an egg-derived virus HAI assay described herein for influenza virus strain A/Brisbane/02/2018. R² is coefficient of determination.

FIG. 1C shows a linear relationship between expected geometric mean titer (GMT) and GMT observed using an egg-derived virus HAI assay described herein for influenza virus strain B/Maryland/15/2016. R² is coefficient of determination.

FIG. 1D shows a linear relationship between expected geometric mean titer (GMT) and GMT observed using an egg-derived virus HAI assay described herein for influenza virus strain B/Phuket/3073/2013. R² is coefficient of determination.

FIG. 2A shows a linear relationship between expected geometric mean titer (GMT) and GMT observed using a virus-like particle (VLP) HAI assay described herein for influenza virus strain A/Kansas/14/2017. R² is coefficient of determination.

FIG. 2B shows a linear relationship between expected geometric mean titer (GMT) and GMT observed using a virus-like particle (VLP) HAI assay described herein for influenza virus strain A/Brisbane/02/2018. R² is coefficient of determination.

FIG. 2C shows a linear relationship between expected geometric mean titer (GMT) and GMT observed using a virus-like particle (VLP) HAI assay described herein for influenza virus strain B/Maryland/15/2016. R² is coefficient of determination.

FIG. 2D shows a linear relationship between expected geometric mean titer (GMT) and GMT observed using a virus-like particle (VLP) HAI assay described herein for influenza virus strain B/Phuket/3073/2013. R² is coefficient of determination.

FIG. 2E shows a linear relationship between expected geometric mean titer (GMT) and GMT observed using a virus-like particle (VLP) HAI assay described herein for influenza virus strain A/California/94/2019. R² is coefficient of determination.

FIG. 2F shows a linear relationship between expected geometric mean titer (GMT) and GMT observed using a virus-like particle (VLP) HAI assay described herein for influenza virus strain A/Cardiff/0508/2019. R² is coefficient of determination.

FIG. 2G shows a linear relationship between expected geometric mean titer (GMT) and GMT observed using a virus-like particle (VLP) HAI assay described herein for influenza virus strain A/Netherlands/1268/2019. R² is coefficient of determination.

FIG. 2H shows a linear relationship between expected geometric mean titer (GMT) and GMT observed using a virus-like particle (VLP) HAI assay described herein for influenza virus strain A/Tokyo/EH1801/2018. R² is coefficient of determination.

FIG. 3A shows a comparison of geometric mean ratio (GMR) for homologous and drifted seasonal strains of clinical samples taken from qNIV-E-301 using duplicate results compared to random titer (singleton). GMR was defined as the ratio of post-vaccination and pre-vaccination HAI GMT's within the same treatment group. CI is confidence interval.

FIG. 3B shows a comparison of seroprotection rate for homologous and drifted seasonal strains of clinical samples taken from qNIV-E-301 using duplicate results compared to random titer (singleton). Seroprotection was defined as a titer of ≥1:40 (a titer that gives a 50% reduction in disease). CI is confidence interval; SPR is seroprotection rate.

FIG. 3C shows a comparison of seroconversion rate for homologous and drifted seasonal strains of clinical samples taken from qNIV-E-301 using duplicate results compared to random titer (singleton). Seroconversion was defined as HAI titer post-vaccination meeting one of the following criteria: either pre-vaccination titer <1:10 and post-vaccination titer ≥1:40, or pre-vaccination titer ≥1:10 and at least a 4-fold increase in post-vaccination titer. CI is confidence interval; SCR is seroconversion rate.

FIG. 4A shows a correlation analysis of the HAI assay described herein a qualified microneutralization (MN) assay for influenza strain A/Brisbane/02/2018. CI is confidence interval; GMT is geometric mean titer; HAI is hemagglutination inhibition; MN is microneutralization; R² is coefficient of determination. The dotted line shows 95% CI.

FIG. 4B shows a correlation analysis of the HAI assay described herein a qualified microneutralization (MN) assay for influenza strain A/Kansas/14/2017. CI is confidence interval; GMT is geometric mean titer; HAI is hemagglutination inhibition; MN is microneutralization; R² is coefficient of determination. The dotted line shows 95% CI.

FIG. 4C shows a correlation analysis of the HAI assay described herein a qualified microneutralization (MN) assay for influenza strain B/Maryland/15/2016. CI is confidence interval; GMT is geometric mean titer; HAI is hemagglutination inhibition; MN is microneutralization; R² is coefficient of determination. The dotted line shows 95% CI.

FIG. 4D shows a correlation analysis of the HAI assay described herein a qualified microneutralization (MN) assay for influenza strain B/Phuket/3073/2013. CI is confidence interval; GMT is geometric mean titer; HAI is hemagglutination inhibition; MN is microneutralization; R² is coefficient of determination. The dotted line shows 95% CI.

FIG. 5 shows a table summarizing the results of egg-derived virus and VLP HAI assay sample inter-assay, intra-assay, and total precision for homologous and drifted seasonal influenza strains. When the HAI titer was <10, a value of 5 was used for calculation purposes. % GCV is percent geometric coefficient of variation; HAI is hemagglutination inhibition; VLP is virus-like particle.

FIG. 6 shows a table summarizing the results of egg-derived virus and VLP HAI assay overall precision for homologous and drifted seasonal influenza strains. In the egg-derived virus HAI assay, overall assay precision was evaluated by testing 46 serum samples for each virus strain, except for A/Kansas/14/2017 for which 56 samples were tested. In the VLP HAI assay, precision was evaluated by testing 46 samples for each homologous VLP strain, and 48 samples for antigenically drifted VLPs. When the HAI titer was <10, a value of 5 was used for calculation purposes. % GCV is percent geometric coefficient of variation; HAI is hemagglutination inhibition; VLP is virus-like particle.

FIG. 7 shows a table summarizing the results of HAI assay subtype/lineage-level specificity for seasonal influenza egg-derived viruses using RBCs for H3N2, H1N1, B/Victoria, and B/Yamagata influenza strains. Geometric mean titer (GMT) was defined as the antilog of the mean of the log-transformed HAI titers for a given treatment group. When the HAI titer was <10, a value of 5 was used for calculation purposes. Bold numbers indicate the HAI results from homologous virus and antiserum pair. GMT is geometric mean titer; HA is hemagglutinin; HAI is hemagglutination inhibition; RBCs is red blood cells.

FIG. 8 shows a table summarizing the results of HAI assay subtype/lineage-level specificity for seasonal influenza VLPs using RBCs for H3N2, H1N1, B/Victoria, and B/Yamagata influenza strains. Geometric mean titer (GMT) was defined as the antilog of the mean of the log-transformed HAI titers for a given treatment group. When the HAI titer was <10, a value of 5 was used for calculation purposes. Bold numbers indicate the HAI results from homologous virus and antiserum pair. GMT is geometric mean titer; HA is hemagglutinin; HAI is hemagglutination inhibition; RBCs is red blood cells.

FIG. 9 shows a table showing overall results for precision (% GCV) for singleton VLP HAI titers and replicate (duplicate) GMT for four homologous influenza strains. % GCV is percent geometric coefficient of variation; GMT is geometric mean titer; HAI is hemagglutination inhibition; VLP is virus like particle.

FIG. 10A shows a table comparing qNIV-E-301 GMT of HAI antibody titers for duplicate and randomly selected titers (singleton) from VLP HAI clinical testing for selected homologous and drifted seasonal influenza strains. 2019-2020 Fluzone® Quadrivalent was used. Geometric mean titer (GMT) was defined as the antilog of the mean of the log-transformed HAI titers for a given treatment group. CI is confidence interval; GMT is geometric mean titer; HAI is hemagglutination inhibition; VLP is virus-like particle.

FIG. 10B shows a table comparing qNIV-E-301 GMT of HAI antibody titers for duplicate and randomly selected titers (singleton) from VLP HAI clinical testing for selected homologous and drifted seasonal influenza strains. 2019-2020 Fluzone® Quadrivalent was used. Geometric mean titer (GMT) was defined as the antilog of the mean of the log-transformed HAI titers for a given treatment group. CI is confidence interval; GMT is geometric mean titer; HAI is hemagglutination inhibition; VLP is virus-like particle.

FIG. 10C shows a table comparing qNIV-E-301 GMT of HAI antibody titers for duplicate and randomly selected titers (singleton) from VLP HAI clinical testing for selected homologous and drifted seasonal influenza strains. 2019-2020 Fluzone® Quadrivalent was used. Geometric mean titer (GMT) was defined as the antilog of the mean of the log-transformed HAI titers for a given treatment group. CI is confidence interval; GMT is geometric mean titer; HAI is hemagglutination inhibition; VLP is virus-like particle.

FIGS. 11A-11E shows a table with the source and details of human and animal serum samples tested in egg-derived virus and VLP HAI validation assays. 1Samples used only in the egg-derived virus HAI assay. 2Samples used in the VLP HAI assay: NIBSC Influenza anti-B/Brisbane/60/2008-HA Serum, Code 15/312; NIBSC Influenza Anti-B/Colorado/06/2017-Like HA Serum, Code 18/170. ³Samples used only in the VLP HAI assay. HA is hemagglutinin; HAI is hemagglutination inhibition; NIBSC is National Institute for Biological Standards and Control; VLP is virus-like particle.

FIGS. 12A-12B shows a table with the results of linearity regression parameters of egg-derived virus HAI assay for four homologous seasonal influenza strains (A/Kansas, A/Brisbane, B/Maryland, and B/Phuket). % GSD is percent geometric standard deviation; HAI is hemagglutination inhibition; LCL is lower confidence limit; N/A is not applicable; R² is coefficient of determination; UCL is upper confidence limit.

FIGS. 13A-13C shows a table with the results of linearity regression parameters of VLP HAI assay for four homologous seasonal influenza strains (A/Kansas, A/Brisbane, B/Maryland, and B/Phuket) and drifted strains (A/California, A/Cardiff, A/Netherlands, and A/Tokyo). % GSD is percent geometric standard deviation; HAI is hemagglutination inhibition; LCL is lower confidence limit; N/A is not applicable; R² is coefficient of determination; UCL is upper confidence limit; VLP is virus-like particle.

FIG. 14 shows a table with the accuracy (% Relative bias) and precision (% GCV) of the HAI titers linearity with A/Kansas/14/2017 egg-derived virus and VLP. % GCV is percent geometric coefficient of variation; GMT is geometric mean titer; HAI is hemagglutination inhibition; VLP is virus-like particle.

FIGS. 15A-15C shows a table with the accuracy (% Relative bias) and precision (% GCV) of the HAI titers linearity with A/Brisbane/02/2018, B/Maryland/15/2016, B/Phuket/3073/2013, A/California/94/2019, A/Cardiff/0508/2019, A/Netherlands/1268/2019, and A/Tokyo/EH1801/2018 virus strains. % GCV is percent geometric coefficient of variation; GMT is geometric mean titer; HAI is hemagglutination inhibition; VLP is virus-like particle.

FIG. 16 shows a table showing assay robustness as the effect of human RBC suspension storage time using for homologous seasonal influenza strains (A/Kansas, A/Brisbane, B/Maryland, and B/Phuket). 1Baseline values were overall HAI GMT from the precision assay runs utilizing fresh 0.75% RBCs stored at 2 to 8° C. for less than a week (≤7 days). GMT is geometric mean titer; HAI is hemagglutinin inhibition; RBC is red blood cells; VLP is virus-like particle.

FIG. 17 shows a table showing assay robustness as the effect of plat reading time (incubation time) using four homologous seasonal influenza strains (A/Kansas, A/Brisbane, B/Maryland, and B/Phuket). GMT is geometric mean titer; HAI is hemagglutinin inhibition; VLP is virus-like particle.

FIG. 18 shows a table showing assay robustness in terms of HAI GMT % difference from baseline assessing the effect of serum/virus (egg-derived virus or VLP) incubation time and plate reading time using four homologous seasonal influenza strains (A/Kansas, A/Brisbane, B/Maryland, and B/Phuket). Geometric mean titer was defined as the antilog of the mean of the log-transformed HAI titers for a given treatment group. Baseline HAI GMTs were from assay runs in which serum/virus (egg-derived virus or VLP) incubation time was 1 h and the plate reading time was 90 min after RBC addition. GMT is geometric mean titer; HAI is hemagglutinin inhibition; VLP is virus-like particle.

FIG. 19 shows a table showing stability of RDE-treated samples in egg-derived and VLP HAI assays using four homologous seasonal influenza strains (A/Kansas, A/Brisbane, B/Maryland, and B/Phuket). GMT is geometric mean titer; HAI is hemagglutinin inhibition; RDE is receptor-destroying enzyme; VLP is virus-like particle.

FIGS. 20A-20C shows a table with the total % GCV for singleton titers, paired replicates, and random replicates for A/Kansas/14/2017 VLP HAI assay. % GCV is percent geometric coefficient of variation; HAI is hemagglutination inhibition; VLP is virus-like particle.

FIGS. 21A-21C shows a table with the total % GCV for singleton titers, paired replicates, and random replicates for A/Brisbane/02/2018 VLP HAI assay. % GCV is percent geometric coefficient of variation; HAI is hemagglutination inhibition; VLP is virus-like particle.

FIGS. 22A-22C shows a table with the total % GCV for singleton titers, paired replicates, and random replicates for B/Maryland/15/2016 VLP HAI assay. % GCV is percent geometric coefficient of variation; HAI is hemagglutination inhibition; VLP is virus-like particle.

FIGS. 23A-23C shows a table with the total % GCV for singleton titers, paired replicates, and random replicates for B/Phuket/3073/2013 VLP HAI assay. % GCV is percent geometric coefficient of variation; HAI is hemagglutination inhibition; VLP is virus-like particle.

FIG. 24 shows the correlation of hemagglutination inhibition titers against A/Kansas/14/2017 in clinical trial sera as determined by assays using wild-type VLPs and egg-grown viruses as agglutinins. A total of 5,281 serum samples from 2,654 subjects in Novavax clinical trial qNIV-E-301 (Shinde, et al.), including approximately equal numbers of recipients of wild-type VLP and egg-grown inactivated virus 2019-20 Northern hemisphere vaccines, were tested in the novel HAI assay using egg-grown virus (X-axis) and cell-based (wild-type) VLP (Y-axis) for A/Kansas/14/2017. Correlation data (Spearman r with 95% CI) analyzed using GraphPad PRISM (v10.2.0) is shown. The slope of the regression line was 1.18.

FIG. 25 shows a table comparing the manual read titers with CypherOne™ hemagglutination analyzer Readout titers.

### DETAILED DESCRIPTION OF THE INVENTION

### Definitions

As used herein, and in the appended claims, the singular forms “a”, “an”, and “the” include plural referents unless the context clearly dictates otherwise. Thus, for example, reference to “a protein” can refer to one protein or to mixtures of such protein, and reference to “the method” includes reference to equivalent steps and/or methods known to those skilled in the art, and so forth.

As used herein, the term “about” or “approximately” when preceding a numerical value indicates the value plus or minus a range of 10%. For example, “about 100” encompasses 90 and 110. When applied to a range, “about” indicates the lower value of the range minus 10%, and the upper value of the range plus 10%. For example, “a range of about 100 to 200” encompasses a range of 90 (lower value-10%) to 220 (upper value+10%).

As used herein, “substantially free” refers to exclusion of a substance (e.g. a compound, polynucleotide, or polypeptide) such that the substance forms the minority percent (e.g., less than 10%, less than 5%, less than 1%, or less than 0.1%) of the sample in which it is contained.

As used herein, the term “influenza virus” used in reference to an hemagglutination inhibition (HAI) assay provided herein refers to a live, modified live (e.g., weakened), or killed influenza virus (e.g., an egg-derived influenza virus) or a virus-like particle comprising an influenza virus hemagglutinin (HA) glycoprotein. An influenza virus herein includes a functional HA glycoprotein. In some embodiments, an influenza virus can include an influenza virus neuraminidase (NA) glycoprotein. In some embodiments, an influenza virus can lack a functional NA glycoprotein (e.g., having no NA glycoprotein, or having an NA glycoprotein without enzymatic function).

As used herein, the term “hemagglutination activity” refers to the process of agglutinating red blood cells (RBCs). “Agglutination” refers to visible clumping together of RBCs. While other molecules can cause agglutination of RBCs, as used herein, hemagglutination activity refers to agglutination of RBCs mediated by influenza virus HA glycoprotein. “Non-specific hemagglutination activity” refers herein to agglutination of RBCs mediated by a factor other than an influenza virus HA (e.g., NA glycoprotein-mediated agglutination).

As used herein, the term “hemagglutination inhibition” (“HAI”) or “inhibition of influenza virus HA glycoprotein hemagglutination activity” refers to a reduction or elimination of hemagglutination activity mediated by influenza virus HA glycoprotein. A new HAI assay described herein can be used to detect and/or quantitate antibodies in a biological sample that can inhibit influenza virus HA protein hemagglutination activity.

As used herein, a “receptor-destroying enzyme” (“RDE”) refers to an enzyme that cleaves sialic acid from glycoproteins and glycolipids.

As used herein, the term “heat-inactivated” or “heat-inactivation” refers to the treatment of a sample with sufficient heat to reduce or remove enzymatic activity (e.g., RDE enzymatic activity), but retain antibody function in the biological sample. Heat-inactivation can be performed at any suitable temperature and time. For example, a biological sample can be heat inactivated at a temperature of about 50° C. to about 60° C. (e.g., about 54° C. to about 58° C.) for about 20 minutes to about 40 minutes (e.g., about 25 minutes to about 35 minutes, or about 28 minutes to about 32 minutes).

As use herein, the term “hemagglutination inhibition titer” (“HAI titer”) refers to the highest tested dilution of a biological sample that achieves hemagglutination inhibition. The HAI titer is generally reported as the dilution factor. For example, if the highest dilution of a sample that achieves hemagglutination inhibition is 1:10, then the HAI titer is 10.

As used herein, a “hemagglutination unit” (“HAg unit”) refers to the lowest concentration of influenza virus that fully agglutinates a 0.75% suspension of human RBCs in Dulbecco's phosphate buffered saline (DPBS). Generally, to determine the concentration of influenza virus that fully agglutinates a 0.75% suspension of human RBCs, serial dilutions of influenza virus are combined with the RBC suspension to determine the lowest concentration that achieves complete agglutination of the RBCs. 4 HAg units is thus an amount of virus that is four times that of a single HAg.

As used herein, an “immunogenic composition” is a composition that comprises an antigen where administration of the composition to a subject results in the development in the subject of a humoral and/or a cellular immune response to the antigen.

As used herein, the term “adjuvant” refers to a compound or substance that, when used in combination with an antigen, augments or otherwise alters or modifies the immune response induced against the antigen. Modification of the immune response may include intensification or broadening the specificity of either or both antibody and cellular immune responses.

The terms “treat,” “treatment,” and “treating,” as used herein, refer to an approach for obtaining beneficial or desired results, for example, clinical results. For the purposes of this disclosure, beneficial or desired results may include inhibiting or suppressing the initiation or progression of an infection or a disease; ameliorating, or reducing the development of, symptoms of an infection or disease; or a combination thereof.

“Prevention,” as used herein, is used interchangeably with “prophylaxis” and can mean complete prevention of an infection or disease, or prevention of the development of symptoms of that infection or disease; a delay in the onset of an infection or disease or its symptoms; or a decrease in the severity of a subsequently developed infection or disease or its symptoms.

As used herein an “effective dose” or “effective amount” refers to an amount of an immunogen sufficient to induce an immune response that reduces at least one symptom of pathogen infection. An effective dose or effective amount may be determined e.g., by measuring amounts of neutralizing secretory and/or serum antibodies, e.g., by plaque neutralization, complement fixation, enzyme-linked immunosorbent (ELISA), or microneutralization assay.

As used herein, the term “vaccine” refers to an immunogenic composition, such as an antigen derived from a pathogen, which is used to induce an immune response against the pathogen. The immune response may include formation of antibodies and/or a cell-mediated response. Depending on context, the term “vaccine” may also refer to a suspension or solution of an antigen that is administered to a subject to produce an immune response. Preferably, a vaccine induces an immune response that is effective at preventing infection from one or more seasonal influenza virus strain.

As used herein, the term “subject” includes humans and other animals. Typically, the subject is a human. For example, the subject may be an adult, a teenager, a child (2 years to 14 years of age), an infant (birth to 2 year), or a neonate (up to 2 months). In particular aspects, the subject can be up to 4 months old, or up to 6 months old. In aspects, the adults can be seniors about 65 years or older, or about 60 years or older. In aspects, the subject can be a pregnant woman or a woman intending to become pregnant. In other aspects, subject can be not a human, such as a non-human primate, for example, a baboon, a chimpanzee, a gorilla, or a macaque. In certain aspects, the subject may be a pet, such as a dog or cat, or livestock, such as poultry or cattle.

As used herein, the term “pharmaceutically acceptable” means being approved by a regulatory agency of a U.S. Federal or a state government or listed in the U.S. Pharmacopeia, European Pharmacopeia or other generally recognized pharmacopeia for use in mammals, and more particularly in humans. These compositions can be useful as a vaccine and/or antigenic compositions for inducing a protective immune response in a vertebrate.

### Methods of Detecting Antibodies

The viral hemagglutinin (HA) surface glycoproteins are key determinants of vaccine efficacy against seasonal circulating strains of influenza [6]. The hemagglutination inhibition (HAI) assay represents the gold standard method for determining an influenza vaccine-elicited immune response and quantitative antibody titers for the influenza virus [7-9]. However, new influenza vaccine development remains challenging due, in part, to traditional, labor-intensive, and cumbersome HAI assay methods involving avian red blood cells (RBCs), complex sample preparation, subjectivity, and low assay throughput. In addition, certain clades of A/H3N2 viruses agglutinate avian RBCs poorly and alternative assays such as virus neutralization assays must be used. A modified HAI assay protocol has been developed to generate more reliable and consistent data using a 0.08% solution of stabilized human RBCs and an assay buffer containing bovine serum albumin (Morokutti, A.; Redlberger-fritz, M.; Nakowitsch, S.; Krenn, B. M.; Wressnigg, N. Validation of the Modified Hemagglutination Inhibition Assay (MHAI), a Robust and Sensitive Serological Test for Analysis of Influenza Virus-Specific Immune Response. J. Clin. Virol. 2013, 56, 323-330.), avoiding partial hemagglutination or irregular shapes of RBC precipitation with avian-origin RBCs. An automated CypherOne™ Hemagglutination Analyzer (InDevR, Boulder, CO, USA) has also been used to determine and provide a visual permanent record of assay results to eliminate the need for tilting of the HAI plate before image interpretation, thereby simplifying the assay and reducing assay turnaround time (Wilson, G.; Ye, Z.; Xie, H.; Vahl, S.; Dawson, E.; Rowlen, K. Automated Interpretation of Influenza Hemagglutination Inhibition (HAI) Assay: Is Plate Tilting Necessary? PLOS ONE 2017, 12, e0179939.). A recent report employed a combination of guinea pig RBCs (instead of avian RBCs), neuraminidase (NA) inhibitor oseltamivir (to prevent NA-mediated agglutination), and CypherOne™ (InDevR, Boulder, CO, USA) HAI plate reader-based automation but focused solely on the HAI assay for H3N2 influenza strains (Sawant, S.; Gurley, S. A.; Overman, R. G.; Sharak, A.; Mudrak, S. V.; Oguiniii, T.; Sempowski, G. D.; Sarzotti-kelsoe, M.; Walter, E. B.; Xie, H.; et al. H3N2 in Fluenza Hemagglutination Inhibition Method Qualification with Data Driven Statistical Methods for Human Clinical Trials. Front. Immunol. 2023, 14, 1155880.). Although all these studies have reported reproducible and reliable results, they do not achieve a single, harmonized high-throughput HAI assay method for both the H1N1 and H3N2 strains as well as B-type viruses.

Provided herein is an improved and objective novel HAI assay that was developed with recombinant virus-like particles (VLPs) or egg-derived virus as agglutinins and human red blood cells (hemagglutination indicator particle) for the detection and/or measurement of anti-influenza HA antibody titers in biological samples (e.g., human serum). In some embodiments, an HAI assay described herein can include the inactivation of neuraminidase-mediated RBC agglutination, e.g., using oseltamivir treatment. In some embodiments, an HAI assay described herein can include an automated image reader-based analysis of hemagglutination. An HAI assay provided herein can have one or more benefits over previously described HAI assays, as evidenced by HAI assay validation described herein. For example, validated HAI results were equivalent for single and duplicate sample testing and correlated well with a qualified live wild-type influenza microneutralization assay, which demonstrate the suitability of this high-throughput novel modified validated HAI assay for evaluating vaccine immunogenicity and efficacy. In addition, the described HAI assay can be used on singleton samples with similar accuracy to duplicate samples, which can provide a benefit of reducing the biological sample volume, reducing laboratory resources, and/or reduce costs needed to analyze HAI titer in a biological sample. In some embodiments, an HAI assay provided herein can also provide a benefit of being able to be performed in a standardized manner for many different influenza strains.

The herein described assay HAI assay was demonstrated to be precise as indicated by the percent geometric coefficient of variation for intra-, inter-, and total assay precision, as well as accurate as evidenced by percent bias measurements. The herein described HAI assay exhibited linearity, specificity for homologous type/subtype strains, and sensitivity with a starting dilution of 1:10, although other starting dilutions may be suitable. Assay robustness and sample stability were demonstrated as a percentage difference compared to reference condition.

Provided herein are methods for determining if a biological sample (e.g., human serum) contains antibodies that inhibit influenza virus HA glycoprotein hemagglutination activity, comprising: (i) reducing or removing non-specific inhibitors of HA from the biological sample to produce a treated sample; (ii) exposing the treated sample to an influenza virus having an HA glycoprotein to produce a sample-virus mixture; (iii) incubating the sample-virus mixture with human red blood cells (RBCs); and (iv) detecting agglutination of the human RBCs; wherein the biological sample contains antibodies that inhibit HA glycoprotein hemagglutination activity if agglutination of the RBCs is not detected.

In some embodiments, a biological sample can be treated with a receptor-destroying enzyme (RDE) to reduce or remove non-specific inhibitors of HA. A biological sample can be incubated with an RDE at a time and temperature sufficient to reduce or remove non-specific inhibitors of HA. For example, a biological sample can be incubated with an RDE at about 37° C. for about 15 hours to about 24 hours (e.g., about 18 hours to about 20 hours). In some embodiments, after being used to reduce or remove non-specific inhibitors of HA, RDE can be heat-inactivated, e.g., at a temperature of about 50° C. to about 60° C. (e.g., about 54° C. to about 58° C.) for about 20 minutes to about 40 minutes (e.g., about 25 minutes to about 35 minutes, or about 28 minutes to about 32 minutes).

In some embodiments, a treated biological sample can be stored prior to use in an HAI assay described herein. A treated biological sample can be stored for up to 2 months (e.g., up to 1 month) at 2° C. to 8° C. before use in an HAI assay without significantly impacting assay results. In some embodiments, a treated biological sample can be stored for up to 2 months at ≤−20° C. (e.g., up to 1 month) before use in an HAI assay without significantly impacting assay results. Surprisingly, even with two freeze/thaw cycles, treated biological samples can be stored for up to 2 months at ≤−20° C. before use in an HAI assay without significantly impacting assay results. In some embodiments, a biological sample that is undiluted (e.g., untreated), can be stored at ≤−20°±10° C. for at least 1 month, even with seven freeze/thaw cycles, before use in an HAI assay without significantly impacting assay results. A significant impact on assay results is generally considered to be more than a 2-fold difference in results relative to a control—in this case, results using a treated biological sample that has been stored for less than 7 days. In some embodiments, the amount of time that biological sample can be stored before use can depend on the influenza virus being used in the assay.

In some embodiments, a biological sample can be diluted, either before or after treatment to reduce or remove non-specific inhibitors of HA, prior to further assay steps. For example, a biological sample can be diluted to reflect a predetermined hemagglutination inhibition titer threshold (e.g., 1:2, 1:4, 1:5, 1:10, or the like). In some embodiments, a biological sample can be serially diluted two or more times (e.g., 2 to 50 times, 2 to 20 times, 2 to 15 times, 3, 4, 5, 6, 7, 8, 9, 10, 11, or 12 times). Serial dilution can be performed at any appropriate ratio (e.g., 1:2, 1:3, 1:4, 1:10, and the like). In some embodiments, a biological sample can first be diluted one or more times to reflect a predetermined hemagglutination inhibition titer threshold (e.g., 1:4, 1:10, or the like), and then be further serially diluted (e.g., 1:2, 1:4, or the like). In some embodiments, a biological sample can be diluted in a buffer, such as Dulbeco's phosphate buffered saline (DPBS), optionally with calcium and/or magnesium.

A treated biological sample, and optionally, dilutions of the treated biological sample, is exposed to an influenza virus having an HA glycoprotein to produce a sample-virus mixture. The influenza virus acts an agglutinin of RBCs. In some embodiments, an influenza virus can be an egg-derived influenza virus. Such an egg-derived virus is typically a weakened virus. Any available egg-derived influenza virus can be used, including, for example, one or more of an A/H3N2 virus (e.g., A/Kansas/14/2017, A/Coatia/10136RV/2023, or the like), an A/H1N1 virus (e.g., A/Brisbane/02/2018, A/Victoria/4897/2022, or the like), a B/Victoria lineage virus (e.g., B/Maryland/15/2016, B/Austria/1359417/2021, or the like), and a B/Yamagata lineage virus (e.g., B/Phuket/3073/2013). Other suitable egg-derived influenza viruses can be any influenza A virus, having any hemagglutinin subtype (H1-H18) and any neuraminidase subtype (N1-N11), or any influenza B virus, from any lineage.

In some embodiments, an influenza virus can be a virus-like particle (VLP). A VLP can be produced in insect cells (e.g., Sf9) infected with baculoviruses bearing cloned genes of influenza HA glycoprotein, NA glycoprotein, and influenza matrix protein (M1). Any suitable HA glycoprotein, NA glycoprotein, and M1 protein can be used. Beneficially, an influenza virus comprising a VLP used in an HAI assay described herein can be used to assess the presence and/or titer of antibodies that inhibit HA glycoprotein hemagglutination activity for both VLPs that are analogous to egg-derived influenza virus strains, as wells as VLPs that are analogous to wild type strains for which there are no analogous egg-derived influenza virus strains. Suitable VLPs can include, for example, VLP analogues of one or more of an A/H3N2 virus (e.g., A/Kansas/14/2017, A/Coatia/10136RV/2023, A/California/94/2019, A/Cardiff/0508/2019, A/Netherlands/1268/2019, A/Tokyo/EH1801/2018, or the like), an A/H1N1 virus (e.g., A/Brisbane/02/2018, A/Victoria/4897/2022, or the like), a B/Victoria lineage virus (e.g., B/Maryland/15/2016, B/Austria/1359417/2021, or the like), and a B/Yamagata lineage virus (e.g., B/Phuket/3073/2013). Other suitable VLPs can be analogous to any influenza A virus, having any hemagglutinin subtype (H1-H18) and any neuraminidase subtype (N1-N11), or any influenza B virus, from any lineage. A method of making such a VLP can be found in Smith, et al. (Development of influenza H7N9 virus like particle (VLP) vaccine: Homologous A/Anhui/1/2013 (H7N9) protection and heterologous A/chicken/Jalisco/CPA1/2012 (H7N3) cross-protection in vaccinated mice challenged with H7N9 virus. Vaccine 2013, 31, 4305-4313).

In some embodiments, an influenza virus (egg-derived or VLP) can be treated with an NA glycoprotein inhibitor, such as oseltamivir, to reduce or prevent NA glycoprotein-mediated RBC agglutination. In some embodiments, oseltamivir can be included in a stock composition of influenza virus at a concentration of about 60 nM to about 100 nM, or about 80 nM. However, in some embodiments, a VLP can exclude a functional NA glycoprotein. That is, in some embodiments, a VLP can have a NA glycoprotein that is enzymatically inactive or have no NA glycoprotein included.

A stock comprising influenza virus can be diluted to achieve about 2 to about 10 (e.g., about 3 to about 5, or about 4) hemagglutination (HAg) units when combined with biological sample to form a sample-virus mixture. In some embodiments, a stock comprising influenza virus can be diluted in a buffer, such as DPBS.

A sample-virus mixture is incubated with human red blood cells (RBCs) prior to detecting agglutination of the RBCs. The human RBCs are preferably in suspension at a concentration of about 0.5% to about 1% (e.g., about 0.75%). Incubation can be done under conditions that promote hemagglutination in the absence of hemagglutination inhibitors. For example, incubation can be performed at a temperature of from about 15° C. to about 30° C. (e.g., about 18° C. to about 28° C., or about 20° C. to about 22° C.) for about 40 minutes to about 80 minutes (e.g., about 50 minutes to about 70 minutes, or about 60 minutes).

Preferably, human RBCs used in an HAI assay provided herein are from Type O blood. In some embodiments, human RBCs can be treated with an anticoagulant, such as K₃EDTA. In some embodiments, human RBCs can be tested to ensure that they agglutinate similarly to previous lots of human RBCs prior to use. For example, RBCs can be tested in an HAI assay using previously tested biological sample(s) (e.g., human serum) to ensure that the results are similar (e.g., within 2-fold) to previous lots of human RBCs. In some embodiments, human RBCs can be tested with negative and/or positive control biological sample(s). A positive control biological sample can comprise, e.g., sheep serum from HA-hyperimmune sheep, human serum that previously showed positive HAI in a previous HAI assay, or the like. A negative control biological sample can comprise, e.g., anti-HA antibody depleted serum (e.g., from a human).

In some embodiments, a human RBC suspension can be stored prior to use in an HAI assay described herein. A human RBC suspension can be stored for up to 2 weeks (e.g., up to 10 days, or up to 1 week) at 2° C. to 8° C. before use in an HAI assay without significantly impacting assay results. A significant impact on assay results is generally considered to be more than a 2-fold difference in results relative to a control—in this case, results using a human RBC suspension that has been used immediately after dilution of RBCs to form the suspension. In some embodiments, the amount of time that a human RBC suspension can be stored before use can depend on the influenza virus being used in the assay.

Following incubation of human RBCs with sample-virus mixture, agglutination of the RBCs is assessed. Preferably, assessment of agglutination is performed 0 to about 90 minutes (e.g., 0 to about 60 minutes, about 15 to about 45 minutes, or about 30 minutes) following the end of incubation, e.g., at about 75 minutes to about 150 minutes, about 80 minutes to about 100 minutes, or about 90 minutes after incubation of human RBCs with sample-virus mixture begins.

Agglutination of human RBCs can be assessed manually, or using an automated image reader-based analysis of hemagglutination, such as the CypherOne™ HAI plate reader marketed by InDevR, Inc. (Boulder, CO, USA). Generally, agglutination is visually assessed by observing whether RBCs sink to the bottom of a suspension, indicating no agglutination, or agglutination inhibition, or form a diffuse network, indicating agglutination. If a container, such as a microtiter plate, has a round or U-shaped bottom, non-agglutinated RBCs can appear as a “dot” on the bottom of the container, while agglutinated RBCs are distributed more evenly in the suspension. In some embodiments, agglutination can be recorded, e.g., by photography and/or by digital imaging (e.g., by an automated image reader). If agglutination is not observed in a suspension containing a sample-virus mixture and human RBCs after a selected incubation time, then the biological sample is determined to contain antibodies that inhibit influenza virus HA glycoprotein hemagglutination activity.

An HAI assay described herein can be performed in any suitable container in which hemagglutination inhibition can be assessed. Preferably, a multi-well (e.g., 96-well), clear assay plate with round or U-shaped well bottoms, such as a 96-well U-bottom Microtiter™ plate marketed by Thermo Fisher Scientific (Waltham, MA, USA) is used. However, other formats and brands can be suitably used with appropriate adjustments to assay volumes and/or recordation/assessment of hemagglutination.

In some embodiments, replicates (e.g., duplicates) of each biological sample to be assessed can be subjected to an HAI assay disclosed herein. However, the disclosed assay provides surprisingly consistent results between samples that have been done in duplicate compared to just a single time.

In some embodiments, an HAI assay provided herein can include a step of determining the hemagglutination inhibition titer of the biological sample. Generally, the hemagglutination inhibition titer of the biological sample can be determined when a biological sample is serially diluted, and RBC agglutination is observed in more dilute samples while RBC agglutination is not observed in less dilute samples. Hemagglutination inhibition titer of the biological sample is determined as the highest dilution factor where RBC agglutination is not observed. Generally, hemagglutination inhibition titer of a biological sample is correlated with the concentration of antibodies that inhibit influenza virus hemagglutinin (HA) glycoprotein hemagglutination activity in the biological sample. It was discovered that an HAI assay described herein hemagglutination inhibition titer of a biological sample correlates well with influenza virus neutralizing antibody titer in the biological sample, as measured by a qualified microneutralization (MN) assay.

### Methods of Assessing or Selecting Immunogenic Compositions

Methods for assessing and/or selecting an immunogenic composition for use as a vaccine are also provided herein. Generally, methods for selecting an immunogenic composition for use as a vaccine include obtaining a plurality of biological samples from subjects that have been administered an immunogenic composition directed to a seasonal influenza virus (e.g., from a medical study); performing an HAI assay described above on the plurality of biological samples to determine hemagglutination inhibition titers of the biological samples; and selecting the immunogenic composition for use as a vaccine if the hemagglutination inhibition titers in the plurality of biological samples meets or exceeds a predetermined threshold.

In some embodiments, the predetermined threshold can be based on the average hemagglutination inhibition titers of a plurality of biological samples. For example, a predetermined threshold can be an average hemagglutination inhibition titer of at least 4 (e.g., at least 5, at least 6, at least 10, or the like). In some embodiments, the predetermined threshold can be based on a certain percentage of samples within a plurality of samples exhibiting a particular hemagglutination inhibition titer. For example, a predetermined threshold can be at least 60% (e.g., at least 70%, or at least 80%) of a plurality of having a hemagglutination inhibition titer of at least (e.g., at least 5, at least 6, at least 10, or the like). In yet other embodiments, a predetermined threshold can be based on an increase in hemagglutination inhibition titer following administration of an immunogenic composition as compared to hemagglutination inhibition titer prior to administration of the immunogenic composition. For example, a predetermined threshold can be an average hemagglutination inhibition titer following administration of an immunogenic composition that is more than two-fold (e.g., 3-fold, 4-fold, 5-fold, or the like) higher than the average hemagglutination inhibition titer prior to administration of the immunogenic composition.

In some embodiments, a method for selecting an immunogenic composition for use as a vaccine can comprise comparing hemagglutination inhibition titers between biological samples from a population of subjects given one immunogenic composition and biological samples from a population of subjects given a different immunogenic composition, and selecting the immunogenic composition that results in higher hemagglutination inhibition titers. In some embodiments, a method for selecting an immunogenic composition can comprise comparing three or more different immunogenic compositions.

In some embodiments, hemagglutination inhibition titer need not be the only criteria used for selection of an immunogenic composition as a vaccine. Other selection criteria can also be considered, such as adverse effects, dosing, peak titer development time, cost, stability, and the like. Immunogenic compositions can differ in the type of antigen, presence or absence of an adjuvant, type of adjuvant, and the like.

### Kits

Kits suitable for performing an HAI assay are also provided herein. An influenza virus comprising an HA glycoprotein (i.e., an egg-derived influenza virus or a VLP) can be combined with one or more other components for use in a method provided herein to form a kit. For example, a kit can comprise an influenza virus and one or more of human RBCs (diluted or undiluted), an RDE, an NA glycoprotein inhibitor (e.g., oseltamivir), a dilution buffer (e.g., DPBS, PBS, or the like), and a multi-well assay plate. Typically, components of a kit are each contained in separate containers to prevent cross-contamination, but packaged together to provide a convenient kit to perform a method provided herein. In some embodiments, a kit can include documentation regarding a protocol and/or validation data, or access to such information (e.g., on a website).

### EXAMPLES

### Example 1

A hemagglutination inhibition (HAI) assay was developed using human red blood cells (RBCs) and either egg-derived influenza virus or virus-like particles (VLPs) as the agglutinin. The assay was tested and validated using human serum samples.

### Materials and Methods

### Seasonal Influenza Viruses and VLPs

The validation of HAI assay for egg-derived influenza viruses was performed using the following four vaccine-homologous 2019-2020 Northern Hemisphere seasonal strains: A/Kansas/14/2017 (A/H3N2), A/Brisbane/02/2018 (A/H1N1), B/Maryland/15/2016 (B/Victoria Lineage), and B/Phuket/3073/2013 (B/Yamagata Lineage). VLPs were produced in Sf9 insect cells infected with baculoviruses bearing cloned genes of influenza HA, neuraminidase (NA), and M1. The membrane-bound VLPs were approximately 100 nM in diameter and morphologically resembled influenza virus particles; the NA was enzymatically active. The VLP HAI assay validation included four recombinant Baculovirus/Sf9-produced VLPs corresponding to the aforementioned homologous seasonal influenza vaccine strains and four VLPs corresponding to the following four antigenically drifted (heterologous) A (H3N2) viruses: A/California/94/2019, A/Cardiff/0508/2019, A/Netherlands/1268/2019, and A/Tokyo/EH1801/2018.

### Human RBCs

Human RBCs (Type O) with K3EDTA as an anticoagulant (Biological Specialty Corporation/BioIVT, Westbury, NY, USA, cat. HUMANRBK3-0101896) were used for the HAI assay. Each new donor's erythrocytes were screened for comparability using a panel of previously tested sera, at least 80% of which must return titers within 2-fold of the prior values for acceptance of the new donor. Each washed and diluted lot of 0.75% red blood cell suspension is tested with negative and positive quality control sera to confirm consistent behavior before use in the assay.

### Test Serum Samples

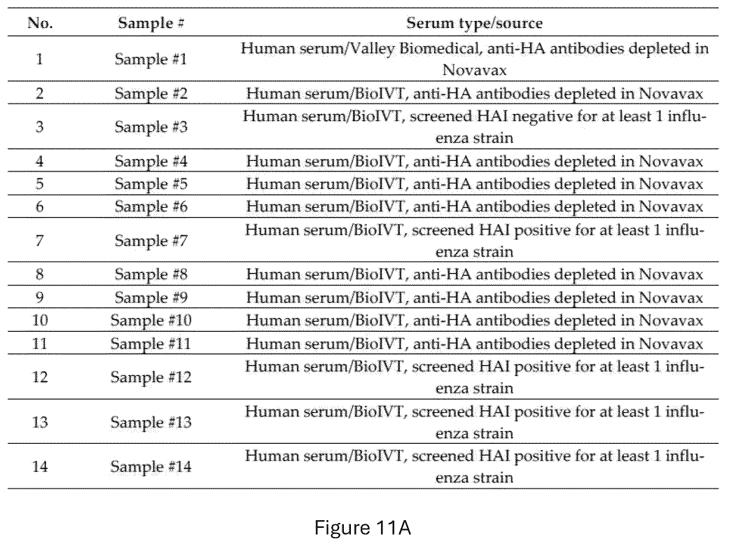

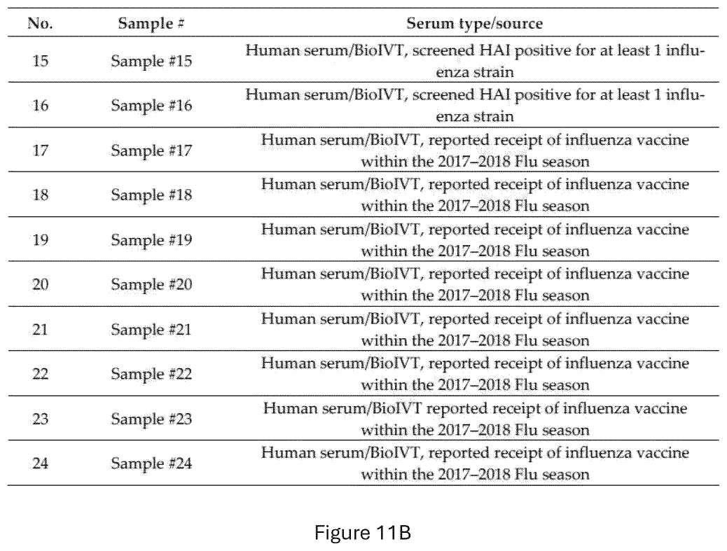

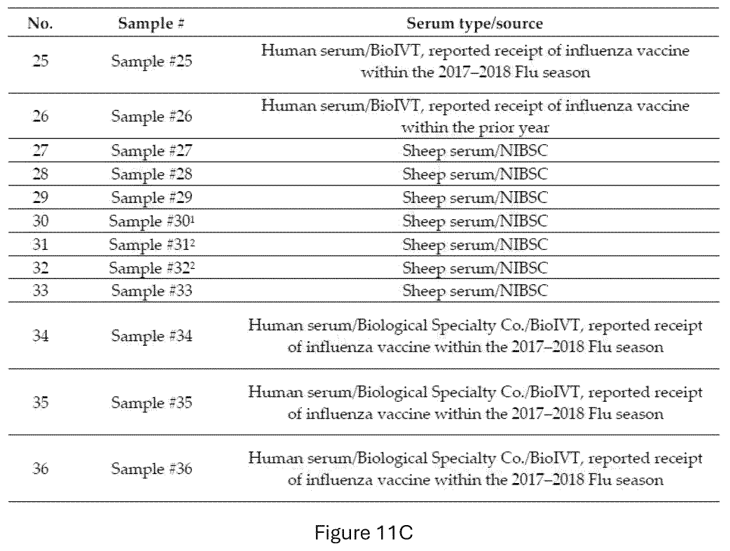

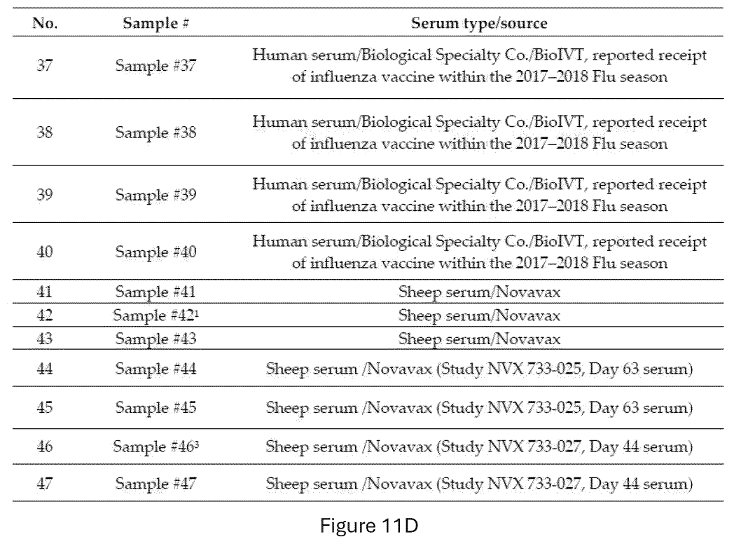

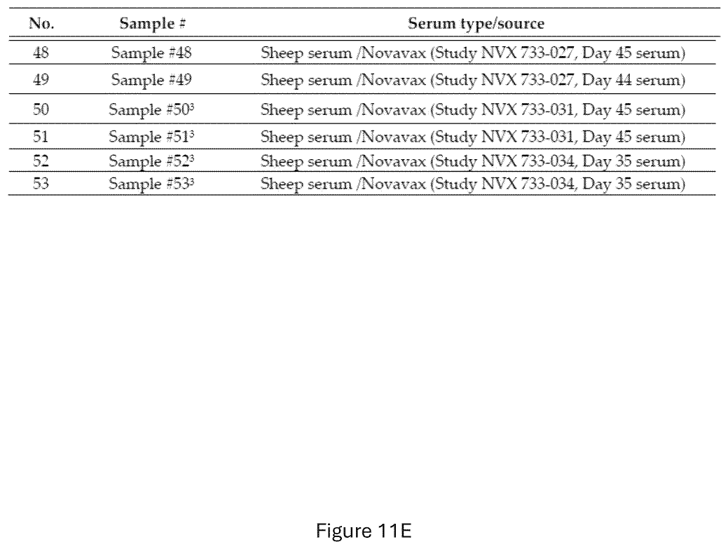

The serum samples used in the assay validation experiments were from healthy humans (BioIVT, Westbury, NY, USA) self-reporting receipt of the influenza vaccine within 1 year before sample collection, human sera that showed positive HAI during the pre-validation serum screening processes, and HA-specific sheep serum samples from virus HA-hyperimmune sheep obtained from the National Institute for Biological Standards and Control and Novavax influenza vaccine non-clinical studies. Negative serum was HA antibody-depleted/stripped human serum (Valley Biomedical, Winchester, VA, USA, cat. HS1200W) diluted 1:2 in PBS. The depletion of HA antibodies from human sera was performed using a negative selection by HA-cross-linked agarose beads (by Novavax, Inc., Gaithersburg, MD, USA). All serum samples used in the assay validation are listed in FIGS. 11A-11E.

### HAI Assay Procedure

The HAI assay was conducted per the WHO Manual for the laboratory diagnosis and virology surveillance of influenza, with some modifications (such as human RBCs and the automated assay readout method mentioned below). To remove nonspecific inhibitors of HA, the serum samples were incubated at 37° C. for 18 to 20 h with a receptor-destroying enzyme (RDE; Denka Seiken Co., Ltd., San Jose, CA, USA, cat. 370013) diluted 1:4 in Dulbecco's Phosphate Buffered Saline with calcium and magnesium (DPBS; Quality Biological, Gaithersburg, MD, USA, cat. 114-059-101), followed by heat-inactivation at 56° C. for 30±2 min and further diluted to 1:10 with DPBS. In the HAI assay, each RDE-treated sample was diluted 1:2 serially in a 96-well U-bottom Microtiter™ plate (Thermo Fisher Scientific, Waltham, MA, USA, cat. 2205) for a total of 10 dilutions with DPBS. The viral stock was adjusted to 4 hemagglutination (HAg) units in DPBS with 80 nM of oseltamivir to prevent possible NA-mediated agglutination (NA inhibitor; ChemScene, Monmouth Junction, NJ, USA, cat. CS-0553). After incubating with 4 HAg units of the virus, the sample-virus mixture was then incubated with 0.75% human RBCs (BioIVT, cat. HUMANRBK3-0101896) for 80 to 100 min at room temperature (RT). To determine serum antibody titers, the HAI assay plates were scored using the automated and validated CypherOne™ Hemagglutination Analyzer (InDevR Inc., Boulder, CO, USA, software versions 3.2.0.0 and 4.0.0.19). The HAI titers were determined from the reciprocal of the highest serum sample dilution that completely prevented hemagglutination.

### Validation Parameters: Precision, Specificity, Linearity, Sensitivity, Assay Robustness, and Sample Stability

The assay parameters evaluated for each vaccine-homologous strain of egg-derived virus and VLP agglutinins were precision, specificity, linearity, sensitivity (lower limit of quantitation [LLOQ]), robustness, and sample stability. The intra-, inter-, and total-assay precision were tested at both the individual sample and strain levels, representing the overall assay variance of all samples tested for each egg-derived virus/VLP strain. Strain-specific sheep sera were used to test the ability of the HAI assay to measure and differentiate the influenza virus type- and subtype-specific antibodies (anti-A/H1N1 [A strain subtype 1 HA], anti-A/H3N2 [A strain subtype 3 HA], and anti-B strain HA antibodies). Negative human serum samples included in the specificity runs were expected to consistently test negative (geometric mean titer [GMT] of 5-7). To determine the assay linearity, for each strain, two influenza HAI-positive samples were tested undiluted and further serially diluted in 1:2 dilution series (from 1:2 up to 1:256) in negative serum, with a target of a minimum of six dilutions above the LLOQ. Samples were tested in replicates in a total of six runs by two analysts over 3 days. The expected HAI titers at each dilution were calculated from the overall HAI GMT from all runs of the undiluted sample divided by the dilution factor. The accuracy of the linearity at each dilution was evaluated in terms of percent relative bias, where values of 100% or −50% correspond to a titer that is 2-fold higher or 2-fold lower, respectively, than the expected titer.

The assay sensitivity was determined in terms of the LLOQ, which is the lowest HAI titer with acceptable precision and accuracy. The LLOQ was determined for samples assayed in the linearity tests. Precision (percent geometric coefficient of variation [% GCV] of HAI titers) and accuracy (percent relative bias) were estimated across the runs for these samples.

Robustness was tested for all egg-derived virus/VLP strains to evaluate the effect of RBC age (storage time from receipt/collection time), serum-agglutinin incubation time, and serum-agglutinin-RBC incubation time (plate reading time) on the HAI results. For the RBC suspension storage time robustness assay, the panel of serum samples were tested using 0.75% RBC stored at 2 to 8° C. for 2 weeks (14±3 days) and 0.75% RBC prepared from 10% RBC suspension stored at 2 to 8° C. for 2 weeks (14±3 days). The HAI GMT of the test storage conditions was compared with the overall HAI GMT from the precision assay runs (utilizing fresh 0.75% RBCs stored at 2 to 8° C. for less than a week [≤7 days]; baseline).

Plate reading time robustness was determined at 75, 90, 120, and 150 min after the addition of the 0.75% RBC suspension to the serum-virus mixture (incubated for 60 min). The HAI GMTs were compared with the standard 90-min reading time. To evaluate the combined serum-agglutinin incubation time and plate reading time robustness, serum-agglutinin incubation time was varied (50, 60, and 70 min) and, for each duration of incubation, the plate reading time was varied (75, 90, 120, and 150 min) after the addition of RBCs. Baseline HAI GMTs were from assay runs in which serum-agglutinin incubation time was 60 min and the plate reading time was 90 min after RBC addition. Additionally, for egg-derived virus/VLP titration and back-titration (virus/VLP titration plate reading time robustness), the plate reading time was determined at 90+10 min following RBC addition.

The stability of RDE-treated samples stored at 2 to 8° C. (1 and 2 months), ≤−20° C. (2 months), and subjected to two freeze/thaw cycles was assessed and compared with the overall HAI GMT from precision runs (baseline; with samples stored at 2 to 8° C. for ≤7 days). The stability of neat (undiluted) serum samples stored at −20±10° C. (1 month) and subjected to seven freeze/thaw cycles (from storage in a −80±10° C. freezer) was assessed using three quality control (QC) samples for each egg-derived virus/VLP strain. The results were compared with the overall GMT from the precision runs (baseline) as described above.

### Singleton (Single Titer) Testing of Serum Samples in the Influenza VLP HAI Assay

To analyze the influence of singleton testing on individual samples (single titer, i.e., singlicate), the validation data of the VLP HAI assay for the four homologous seasonal vaccine strains were used and reproduced. The GMT and % GCV were determined for the validation dataset of each vaccine-homologous strain. For each serum sample, the GMT and % GCV were determined for (1) singleton titers (N=24), (2) paired replicate (duplicate) GMT (N=12), and (3) random titer replicates (duplicate) GMT (N=12). Random titer pairings were intended to simulate potential titer pairings based on the data collected in the validation studies.

The percentage difference in GMT for randomly chosen replicates (random replicate 1 and random replicate 2) was determined relative to the GMT of paired replicates as shown in Equation 1 below.

$$\% Difference = 100 \times \frac{( Random HAI GMT - Paired HAI GMT )}{Paired HAI GMT} \qquad Equation 1$$

For assessing the influence of singleton results and paired duplicates on precision (% GCV), 36 serum samples were tested for the four vaccine-homologous strains. The % difference for % GCV of singleton titers and random replicates was calculated relative to the % GCV of paired replicates as shown in Equation 2 below.

$$\% Difference = 100 \times \frac{( Paired or Random \% GCV - Singleton \% GCV )}{Singleton \% GCV} \qquad Equation 2$$

For evaluating the influence of singleton testing on clinical study results, HAI anti-body titer data corresponding to VLPs from the Phase 3 clinical study qNIV-E-301 (Shinde, V.; Cho, I.; Plested, J. S.; Agrawal, S.; Fiske, J.; Cai, R.; Zhou, H.; Pham, X.; Zhu, M.; Cloney-Clark, S.; et al. Comparison of the Safety and Immunogenicity of a Novel Matrix-M-Adjuvanted Nanoparticle Influenza Vaccine with a Quadrivalent Seasonal Influenza Vaccine in Older Adults: A Randomized Controlled Trial. Lancet Infect. Dis. 2022, 22, 73-84) were utilized. Aside from the aforementioned four vaccine-homologous seasonal influenza strains and the four heterologous viruses, this clinical study included three additional heterologous wild-type strains (A/South Australia/34/2019, A/Idaho/13/2018, and B/Washington/02/2019). Antibody titers using VLP HAI obtained in duplicate (n=2) from approximately 1286 test subjects and two study protocol arms (2019-2020 Fluzone® Quadrivalent and Quad-NIV) were used to model the influence of reporting single titer (n=1) results (randomly selected from one of the paired duplicates) instead of the GMT of paired duplicate (n=2) values. The per-protocol population included 1279 to 1286 test subjects (two time points: Day 0 [pre-vaccination] and Day 28 [post-vaccination]; two vaccination groups: 2019-2020 Fluzone Quadrivalent and Quad-NIV). The GMT (by strain and vaccine group) was calculated as the reported GMT (duplicate) values of the antibody titers using wild-type sequence VLP-HAI. The results of GMT calculated using paired replicates (duplicates) were compared with the GMT calculated using individual titers that were randomly selected. The randomly selected titer GMT results were determined twice (Random titers 1 and 2). The geometric mean ratio (GMR; post-/pre-vaccination) were determined using the reported GMT from paired duplicate samples and randomly selected single titers. The random titer pairings included were intended to simulate potential titer pairings based on the data collected in clinical studies.

The percent seroprotection rate (% SPR) was determined using data from the study qNIV-E-301. The percentage of results with titer ≥1:40 was determined using the reported GMT from paired duplicate samples and using randomly selected single titers (Random 1 and Random 2) for subject visits on Day 28. The percent seroconversion rate (% SCR) was also determined using qNIV-E-301 study data. The results (%) for subject visits on Day 0 (unvaccinated) and Day 28 (vaccinated) where the titer or GMT on Day 28 was higher by ≥4-fold were determined using the reported GMT from paired duplicate samples and using randomly selected single titers (Random 1 and Random 2).

### Correlation Analysis of the HAI Assay and the MN Assay

Serum samples were assessed in the HAI assay (using the same methodology as for validation) and in a qualified influenza MN assay for each indicated wild-type strain (A/Brisbane/02/2018, A/Kansas/14/2017, and B/Maryland/15/2016). The results from the final titers of both assays were compared to determine the correlation between them.

### Statistical Analysis

Statistical analyses of the validation results were performed using SAS® version 9.4 (SAS Institute Inc., Cary, NC, USA) in a Windows (Microsoft Corp., Redmond, WA, USA) environment. The intra- and inter-assay precision were assessed by determining the % GCV through the variance component analysis with the sample as a fixed effect and analyst and day as random effects. The correlation between the HAI and MN assay results was determined by performing linear regression analysis using GraphPad Prism® software version 9.3.1 (GraphPad Software, San Diego, CA, USA).

### Results

### Assay Validation Parameters

### Precision

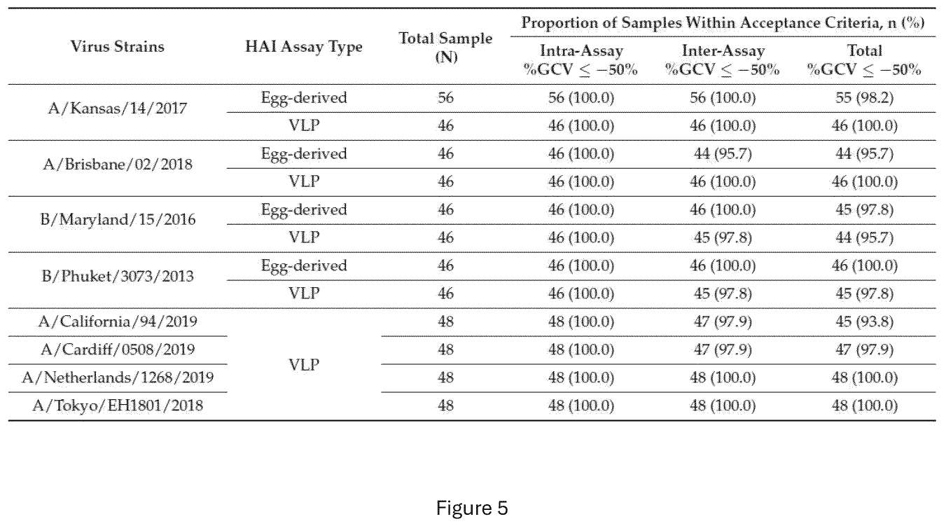

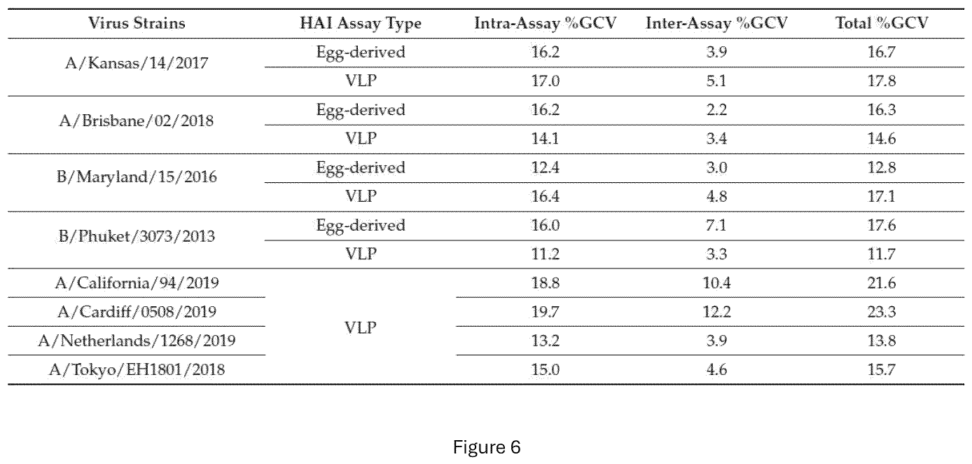

For the egg-derived HAI assay, all four seasonal influenza homologous virus strains met the acceptance criteria of intra-, inter-, and total-assay precision≤50% GCV for at least 80% of the samples tested for each strain (A/Kansas/14/2017:98.2% of the samples, A/Brisbane/02/2018:95.7%, B/Maryland/15/2016:97.8%, B/Phuket/3073/2013: 100%) (FIG. 5). Similarly, for the VLP HAI assays, intra-, inter-, and total-assay precision were ≤50% GCV for 100% of the samples for A/Kansas/14/2017, A/Brisbane/02/2018, A/Netherlands/1268/2019, and A/Tokyo/EH1801/2018 strains; and >93% of the samples for B/Maryland/15/2016, B/Phuket/3073/2013, A/California/94/2019, and A/Cardiff/0508/2019 strains. Additionally, all egg-derived virus/VLP strains met the acceptance criteria of ≤30% GCV for the overall intra-, inter-, and total-assay precision (FIG. 6).

### Specificity

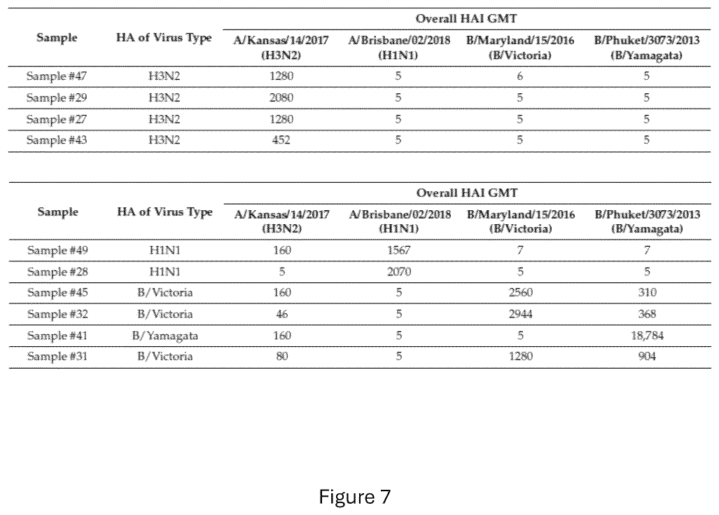

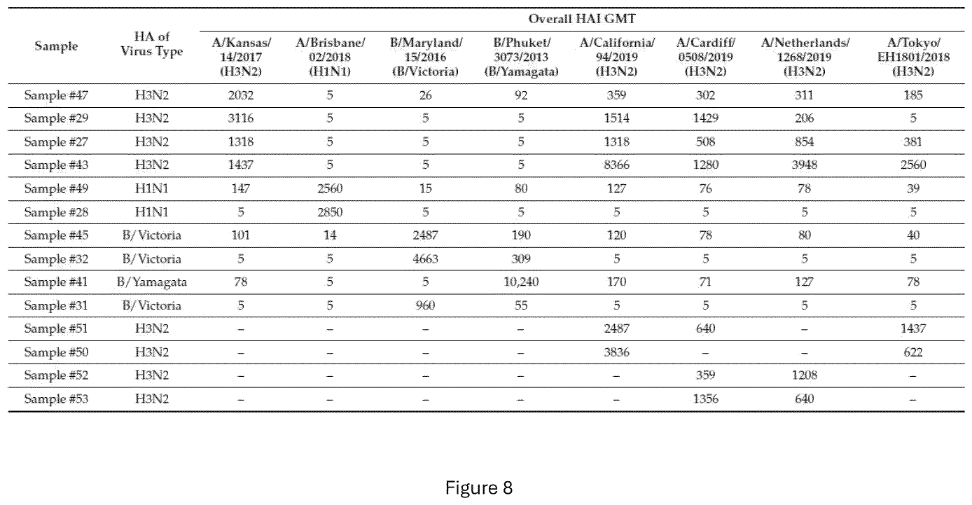

In the specificity analysis, the overall HAI GMT from the strain-homologous immune serum was ≥4-fold higher than from the heterologous serum, thereby meeting the acceptance criterion for both egg-derived and VLP HAI assays. For the egg-derived assay, the overall HAI GMT of subtype/lineage homologous serum was at least 8-fold higher than that of the heterologous serum (A/Kansas/14/2017:8-fold, A/Brisbane/02/2018:300-fold, B/Maryland/15/2016:500-fold, B/Phuket/3073/2013:20-fold; FIG. 7). For the VLP HAI assay, the overall HAI GMT of the subtype/lineage homologous serum was ≥4-fold higher than the heterologous serum HAI GMT (A/Kansas/14/2017:13-fold, A/Brisbane/02/2018:180-fold, B/Maryland/15/2016:95-fold, B/Phuket/3073/2013:33-fold, A/California/94/2019:22-fold, A/Cardiff/0508/2019:17-fold, A/Netherlands/1268/2019:9-fold, A/Tokyo/EH1801/2018:18-fold; FIG. 8). The heterologous sera were either HAI-negative (i.e., HAI GMT of 5) or had low HAI titers compared to the homologous sera. Negative control human serum samples (depleted of anti-HA antibodies) consistently tested negative (GMT of 5).

### Linearity and LLOQ

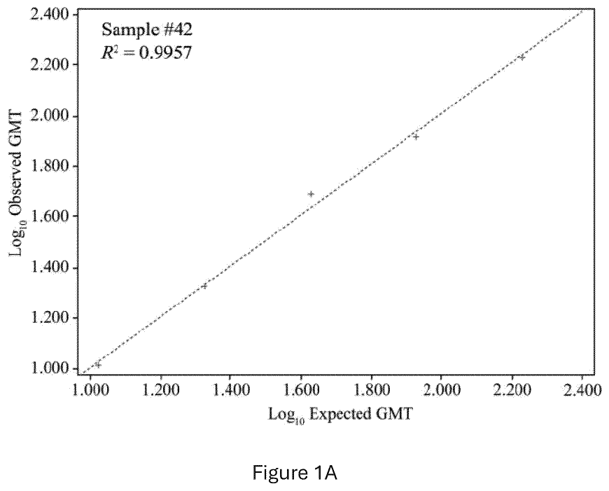

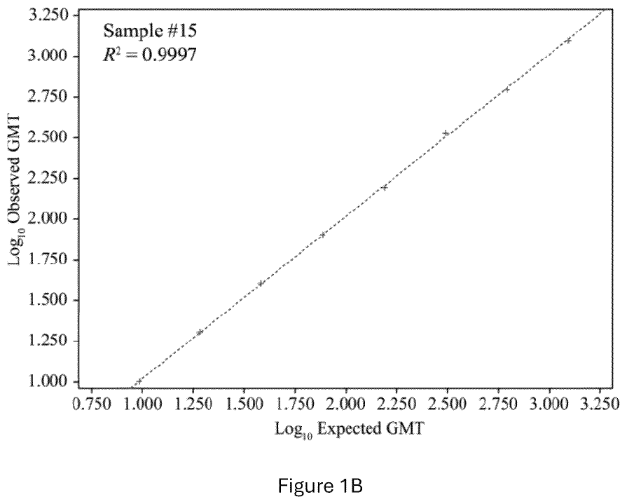

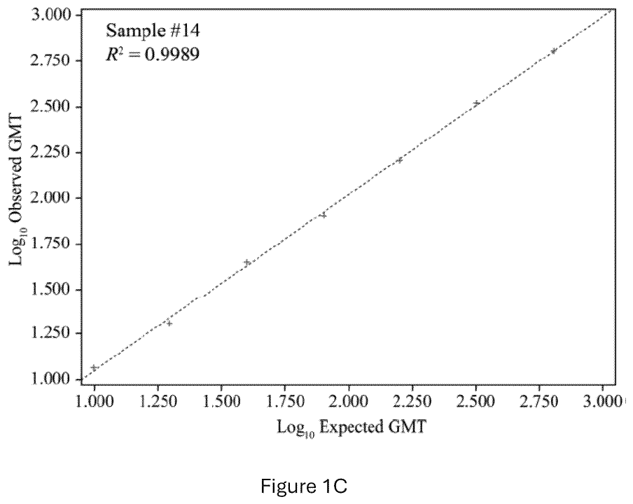

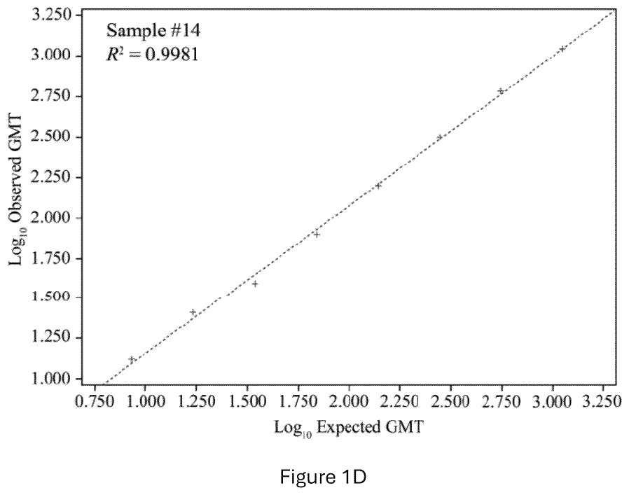

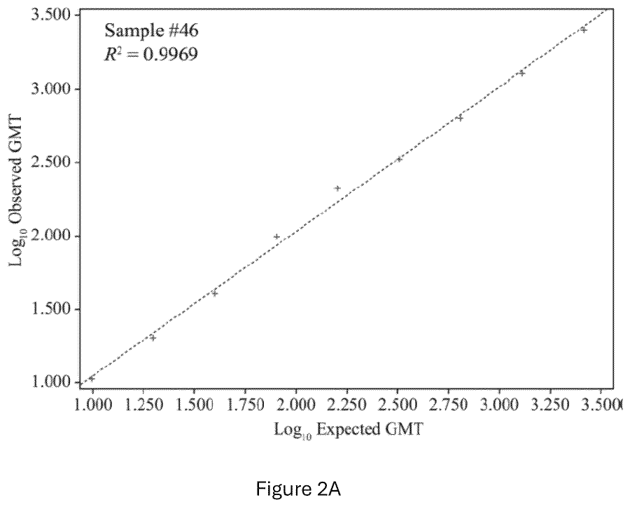

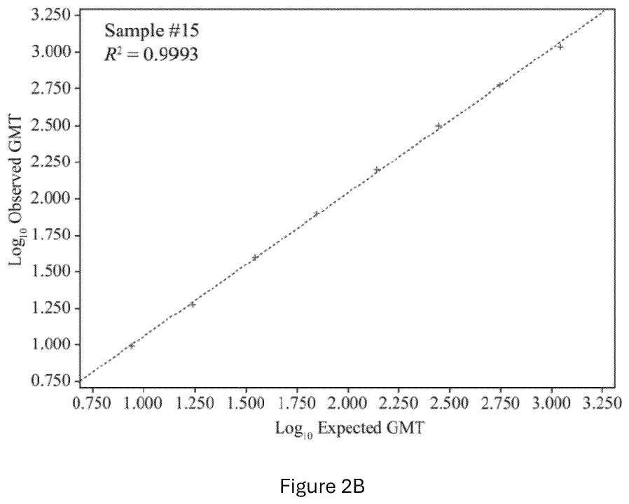

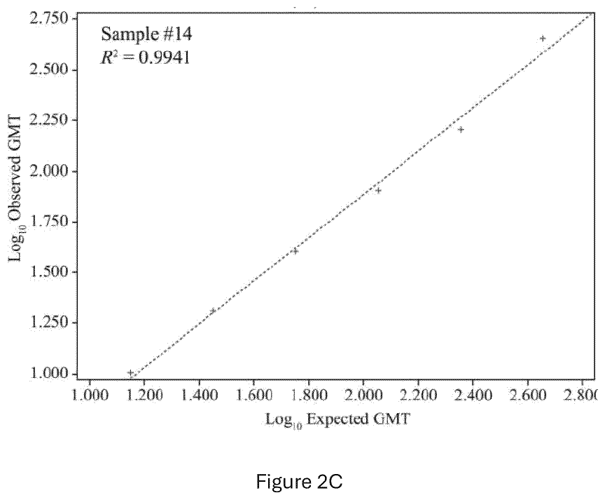

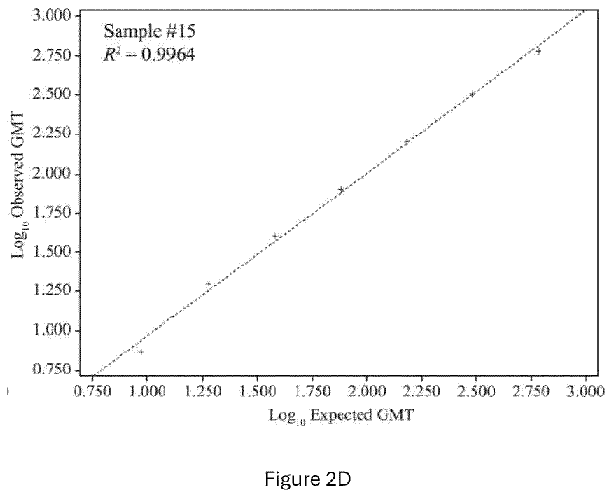

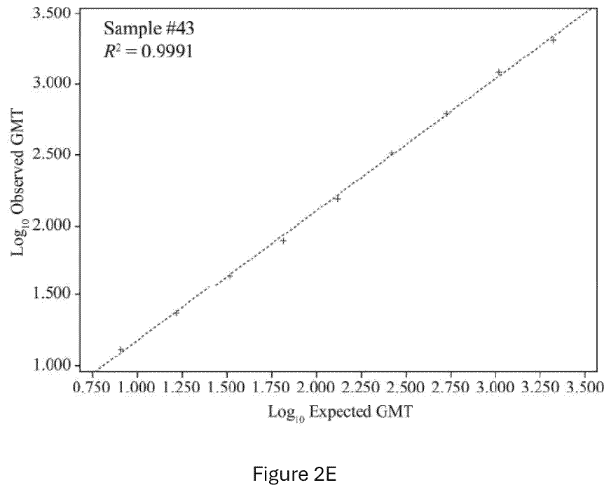

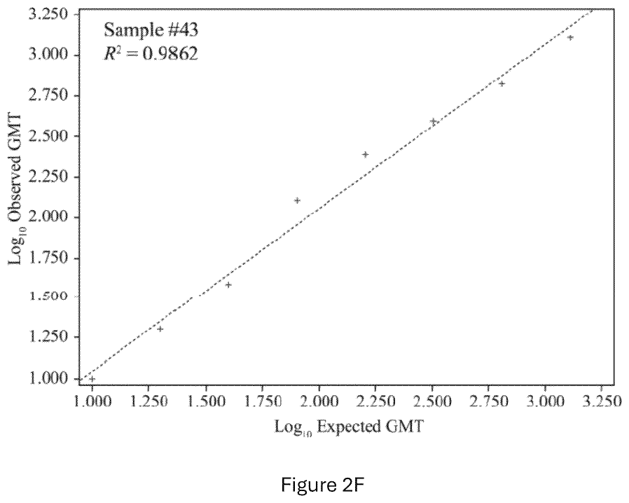

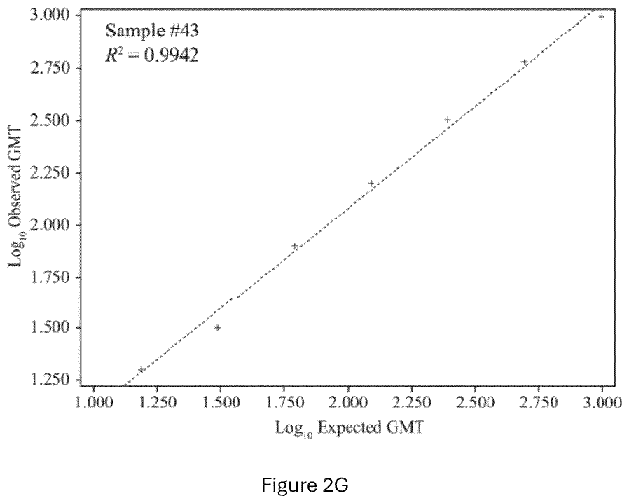

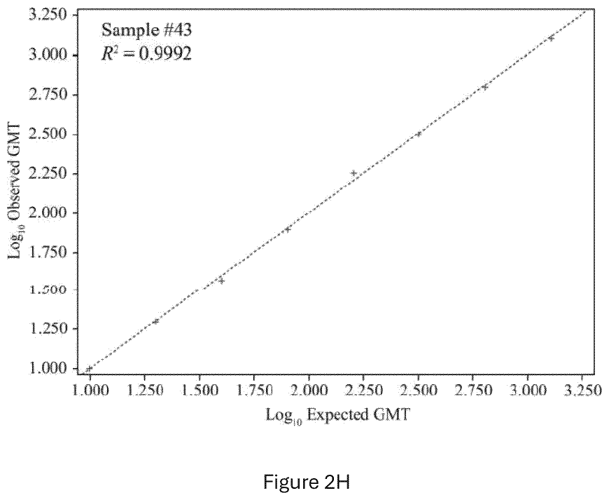

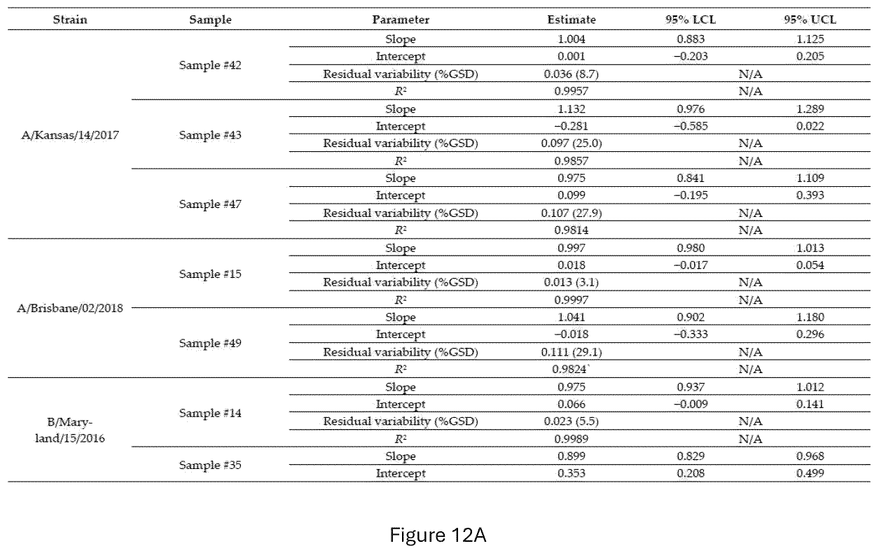

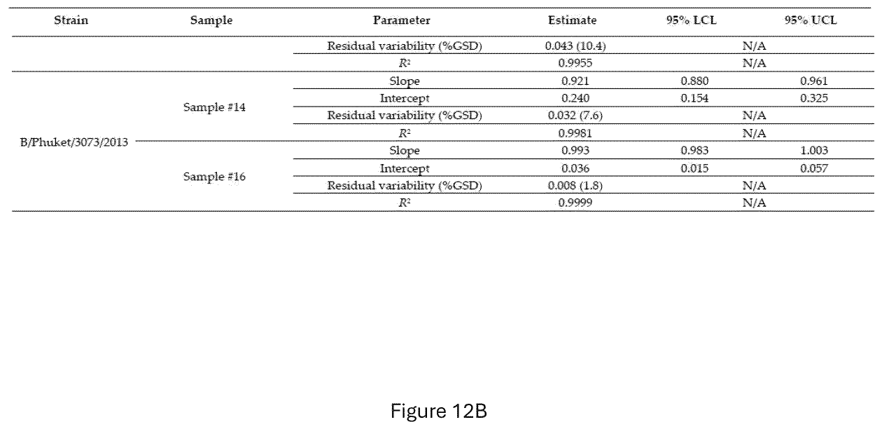

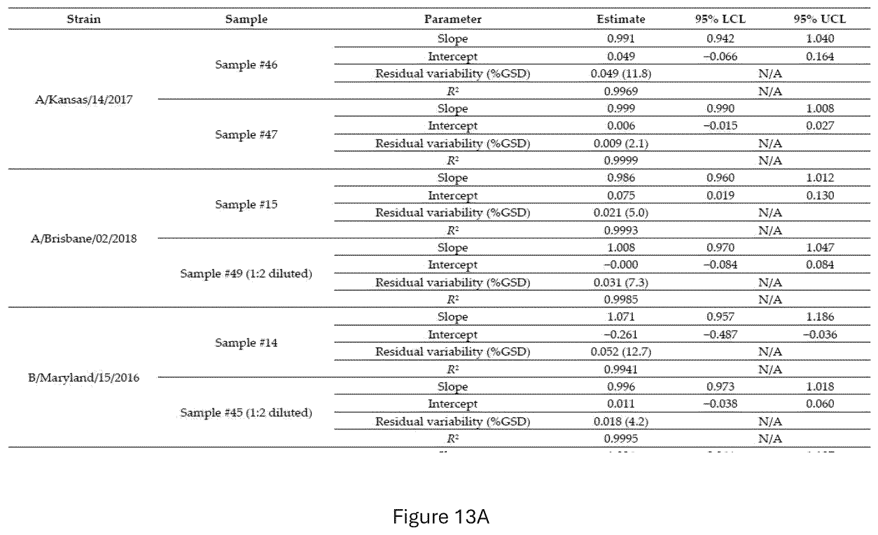

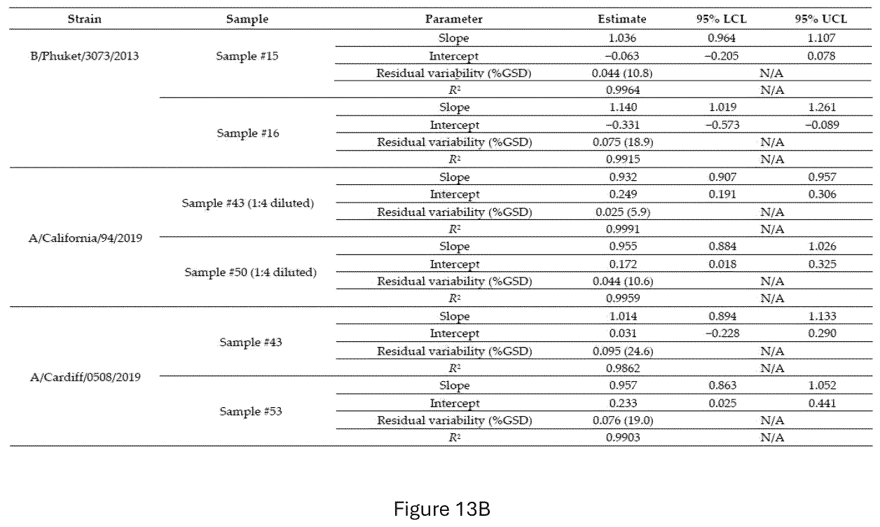

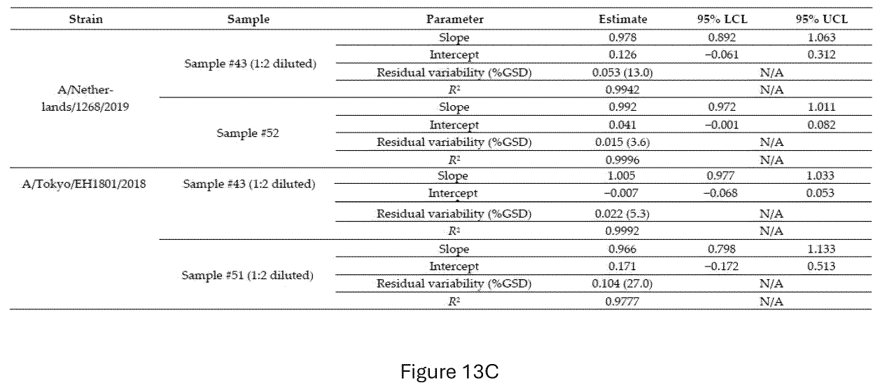

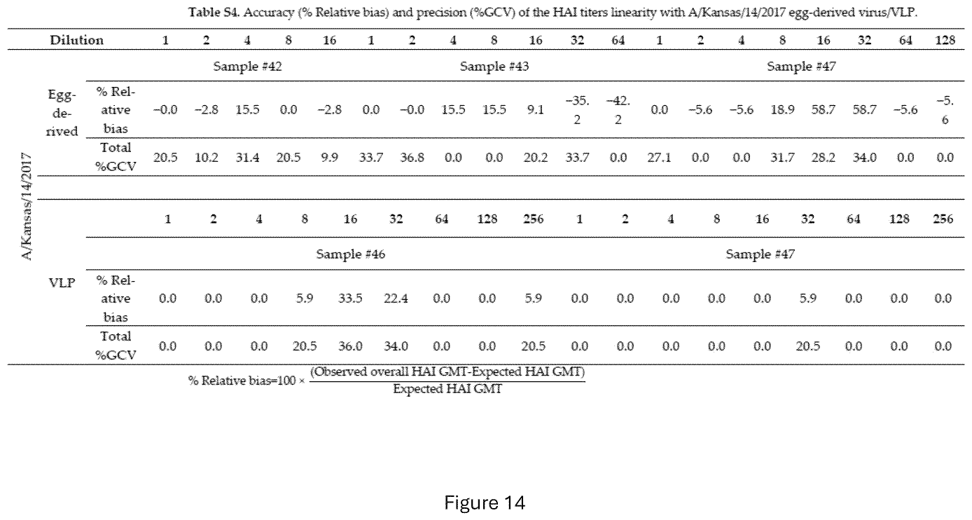

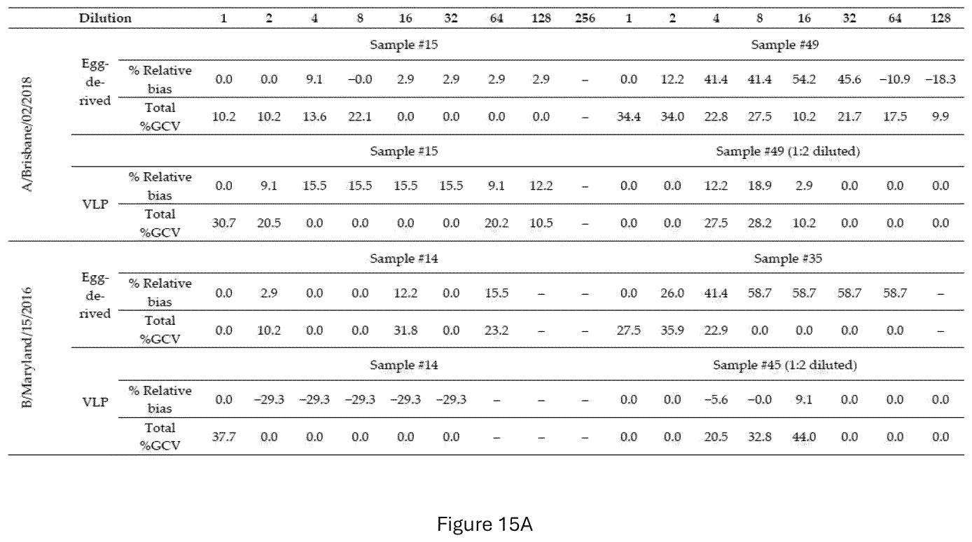

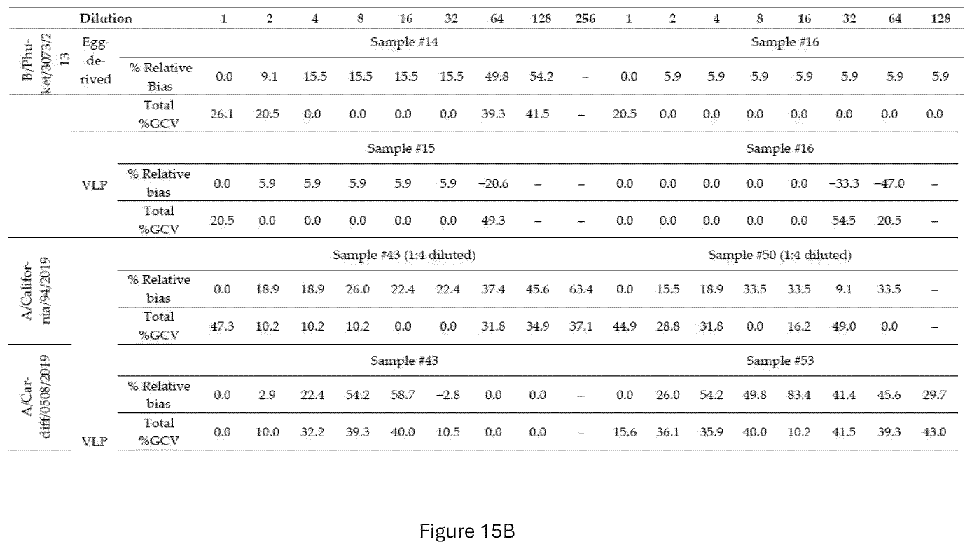

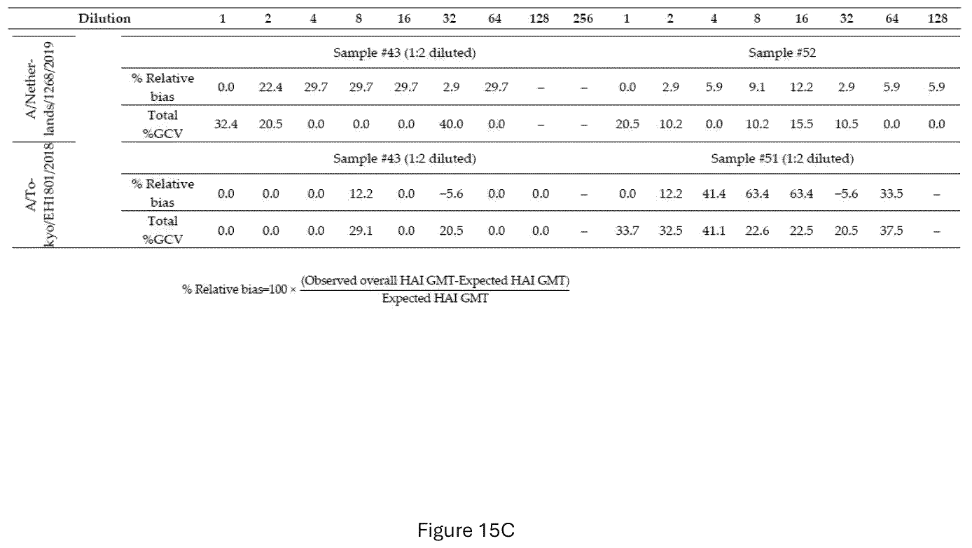

All the virus strain samples with an expected HAI GMT of ≥8 were included in the linear regression and percent relative bias analyses. The linearity of both the egg-derived HAI and VLP HAI assays was successfully demonstrated, with R² ranging from 0.9814 to 0.9999 and from 0.9777 to 0.9999, respectively, for all individual samples examined for each egg-derived virus or VLP agglutinin, meeting the acceptance criterion (regression line R2≥0.95) (FIGS. 1A-1D, 2A-2H, 12A-12B, and 13A-13C). Also, the percent relative bias for all samples (for both the egg-derived virus and VLP agglutinins) was in the acceptance range (−50% to 100% of the expected HAI GMT). In the egg-derived HAI, the % GCV of all samples was <50%. In the VLP HAI, the % GCV was <50% for all the sample dilutions, except for one sample dilution for B/Phuket/3073/2013 VLP (% GCV of 54.5) (acceptance criteria: % GCV≤−60% (FIGS. 14 and 15A-15C).

All samples were diluted to an HAI titer of 10, the lowest titer (GMT) that met the acceptance criteria of intra-, inter-, and total-assay % GCV≤60% and percent relative bias of between-50 and 100. Hence, LLOQ was set at 10 for the egg-derived virus/VLP HAI assay.

### Robustness

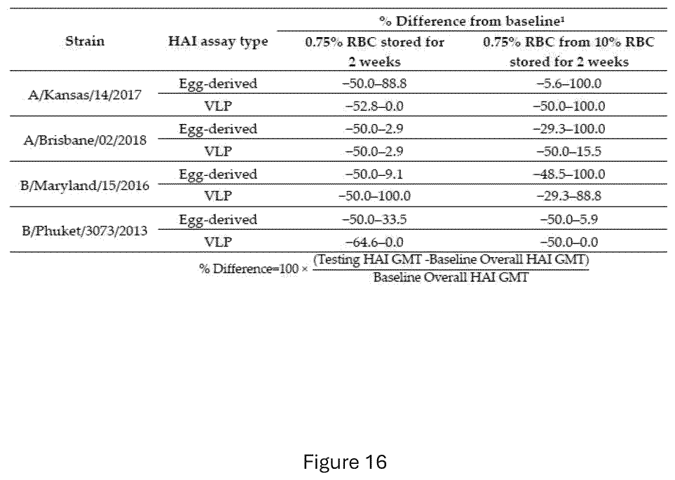

Robustness analysis of the RBC suspension storage time (2 weeks) for the egg-derived virus HAI showed that all samples (100%) tested for all four virus strains exhibited an HAI GMT % difference ranging from −50% to 100% (2-fold difference) compared to the baseline. These findings suggested that RBC suspension can be stored at 2 to 8° C. for up to 2 weeks before use (acceptance criteria: at least 80% of samples with HAI GMT within the 2-fold difference of baseline). In the VLP HAI assay, the majority of the samples (except three) tested for all four VLP strains had a HAI GMT % difference within the range of −50% to 100% compared to the baseline. However, three of the four strains showed an asymmetric tendency to yield a predominance of −50% to 0% change, suggesting the suitability of RBC suspension storage conditions of 2 to 8° C. for a preferred maximum of 1 week (7 days) before use (FIG. 16).

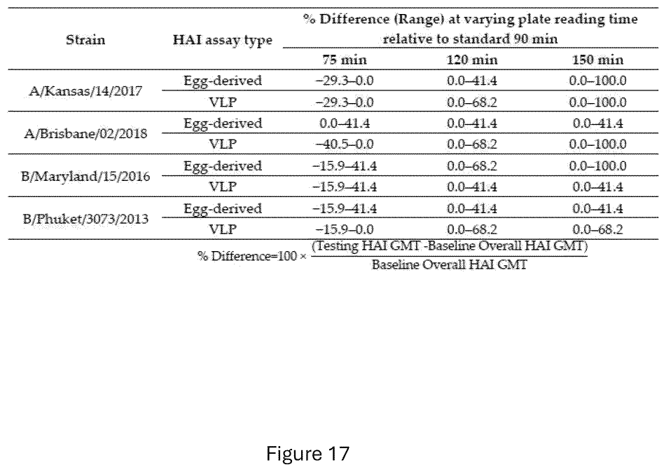

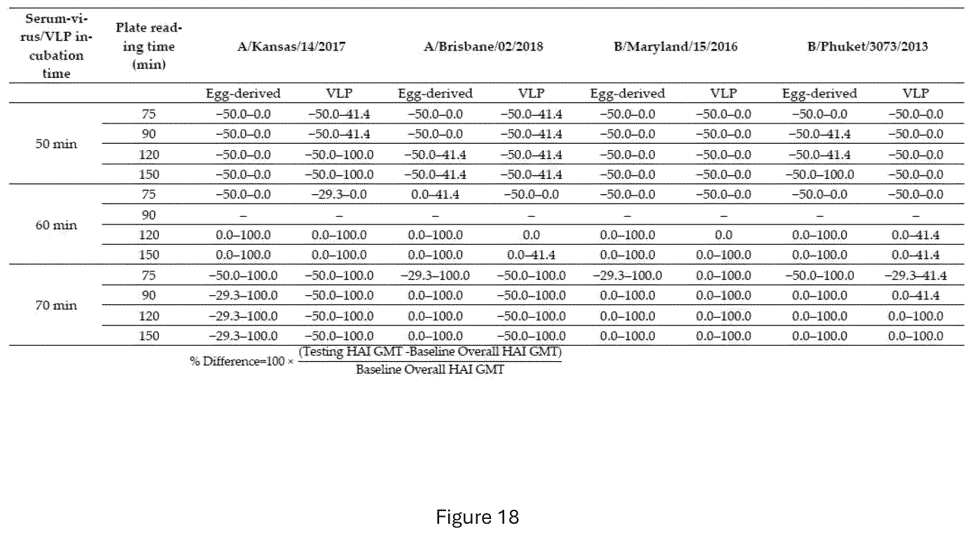

Robustness analysis of the plate reading time (75-, 120-, and 150-min) demonstrated an HAI GMT % difference within the range of −50% to 100% compared to the 90 min plate reading reference, suggesting that, in both the egg-derived virus/VLP HAI assays, the plates can be read within a range of 75 min and 150 min after RBC addition (FIG. 17). In the combined robustness analysis of serum-virus/VLP incubation time and plate reading time, the HAI GMT % difference of all sample HAI titers read at 75, 90, 120, and 150 min after 50 min to 70 min of egg-derived virus/VLP plus serum incubation were within 2-fold (−50% to 100%) of the baseline reading (FIG. 18). However, HAI titers showed more variations when longer incubation times were combined (such as 70 min of egg-derived virus/VLP-serum incubation and 150 min of egg-derived virus/VLP-serum-RBC incubation [plate reading time]), although the HAI GMT % difference was still within 2-fold of the baseline. These findings suggest that experimental conditions with egg-derived virus/VLP-serum incubation time between 50 and 60 min, as well as a plate reading time of 75 to 120 min, can be considered as best practice to maintain result consistency in HAI assays (FIG. 18). Furthermore, the robustness of the plate reading time was also evaluated for 90+10 min after RBC incubation for virus titration and back-titration. Consistent HAI titers were observed at 80 and 100 min post-RBC addition (in line with those at standard 90 min), suggesting that both the egg-derived virus and VLP HAI assay titration plates can be read at 90+10 min of RBC incubation.

### Stability

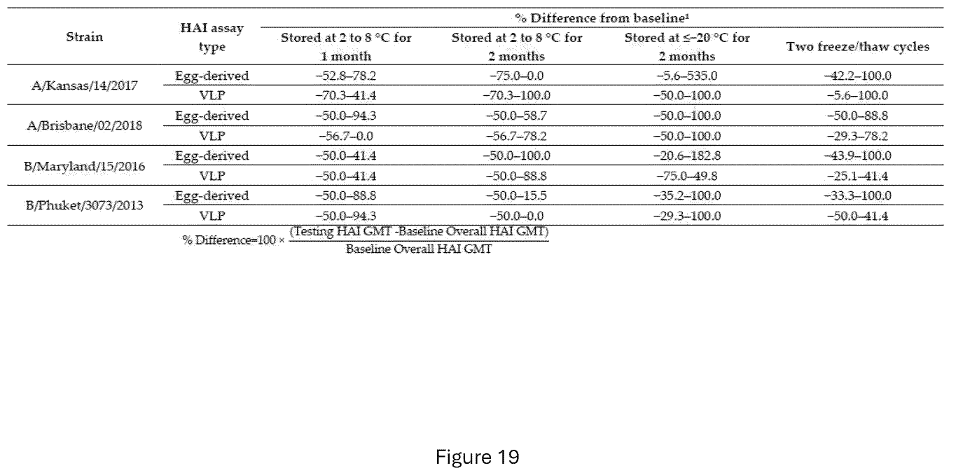

In the RDE-treated sample stability analysis, HAI GMTs of at least 80% of the samples were within the 2-fold difference of the baseline (−50% to 100%) following storage at 2 to 8° C. for 1 month and 2 months, storage at ≤−20° C. for 2 months, and after undergoing two freeze/thaw cycles in both HAI assays (FIG. 19). However, in the egg-derived virus HAI assay, the HAI GMT of samples with 2 months of storage showed increasing variability relative to the baseline and a trend towards decreasing titers compared to 1 month of storage, especially for the A/Kansas/14/2017 and B/Phuket/3073/2013 assays. These findings suggest that to maintain consistency for both egg-derived virus and VLP HAI assays, the storage of RDE-treated samples at 2 to 8° C. should be limited to 1 month prior to testing (FIG. 19).

Stability analysis of the neat (undiluted) serum samples was included to assess sample stability under the potential storage conditions commonly observed in clinical trials. The HAI GMTs of all samples were within the range of −50% to 100% difference of the baseline following seven freeze/thaw cycles and 1 month of storage in a −20° C. freezer, suggesting that the serum samples remained suitable for both the egg-derived virus/VLP HAI assay testing even after undergoing these conditions.

### Influence of Singleton (Single Titer) Sample Testing on GMT of Serum Samples

The GMT of the singleton titers and paired replicates were identical for all samples with all four homologous seasonal influenza strains. Also, there was no significant difference in the GMT of randomly combined sample pairs compared to that of the singleton or paired titers. The randomly chosen replicates showed a % GMT difference of −2.85% to 5.95%, −2.85% to 9.05%, −5.61% to 2.93%, and −5.61% to 5.95% compared to that of the paired replicates for A/Kansas/14/2017, A/Brisbane/02/2018, B/Phuket/3073/2013, and B/Maryland/15/2016, respectively. These differences in GMT were minimal and not significant (within the acceptable 2-fold assay variability). Thus, the random pairing of titer results does not significantly impact the GMT of either the paired or individual titers when assessed at the sample level.

### Influence of Singleton (Single Titer) Results and Paired Duplicates (Mean of Two Titers) on Precision (% GCV) at the Serum Sample Level

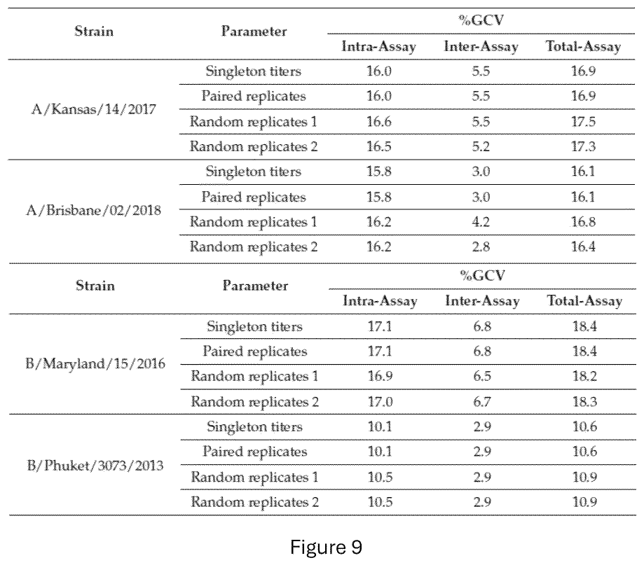

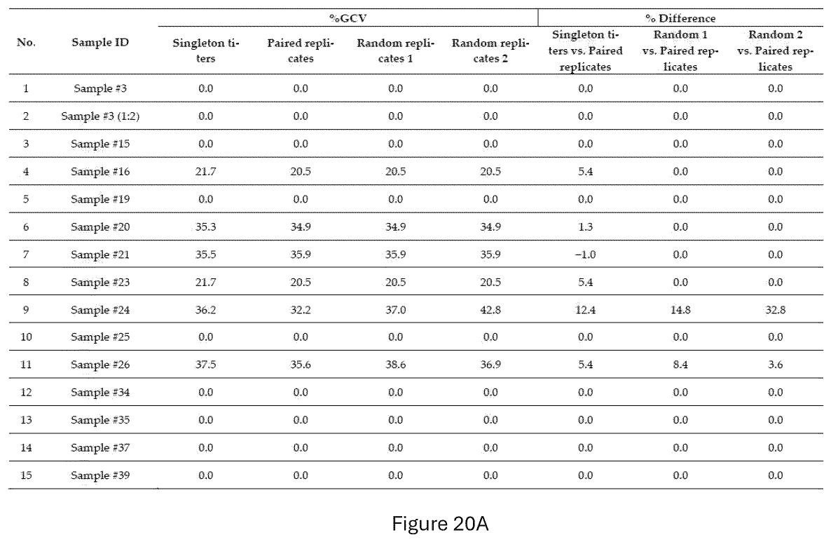

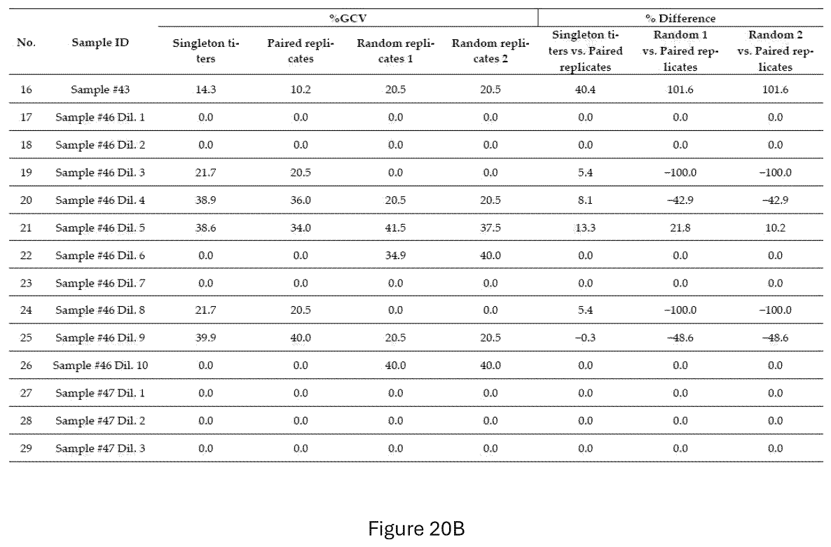

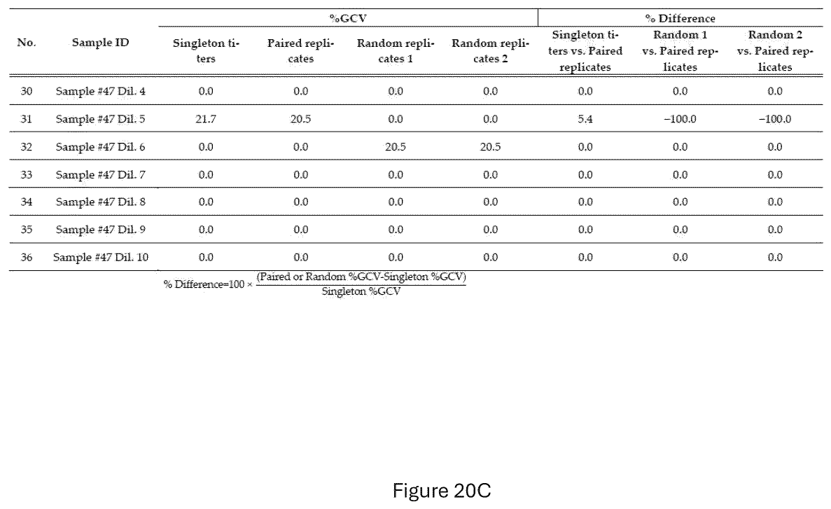

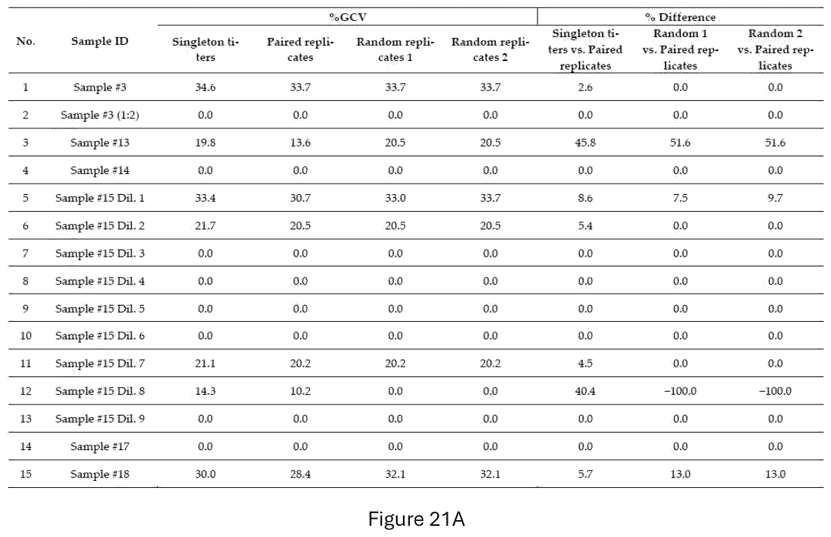

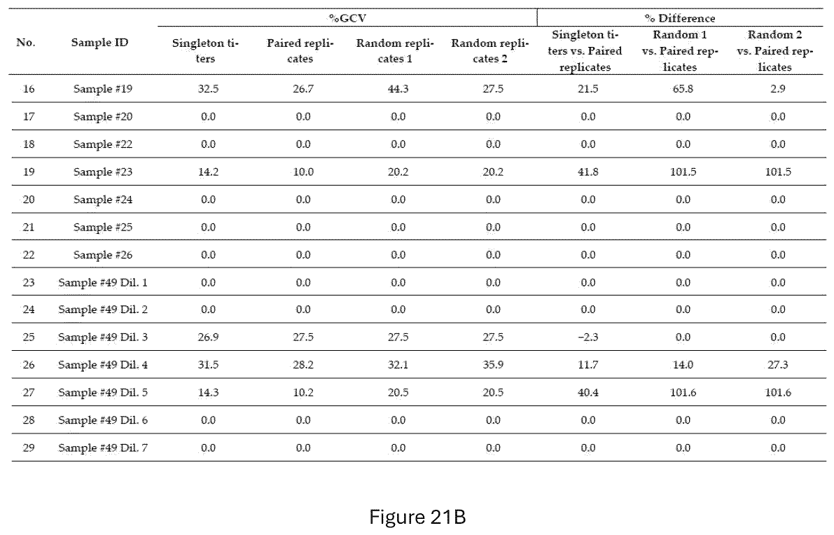

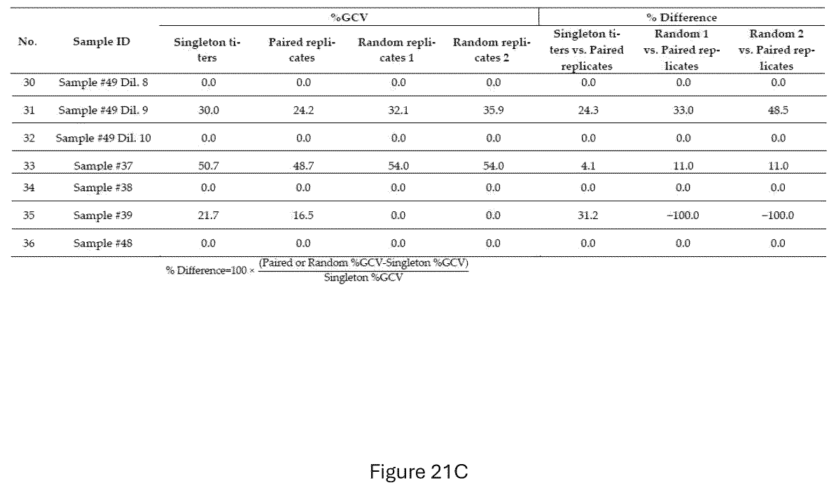

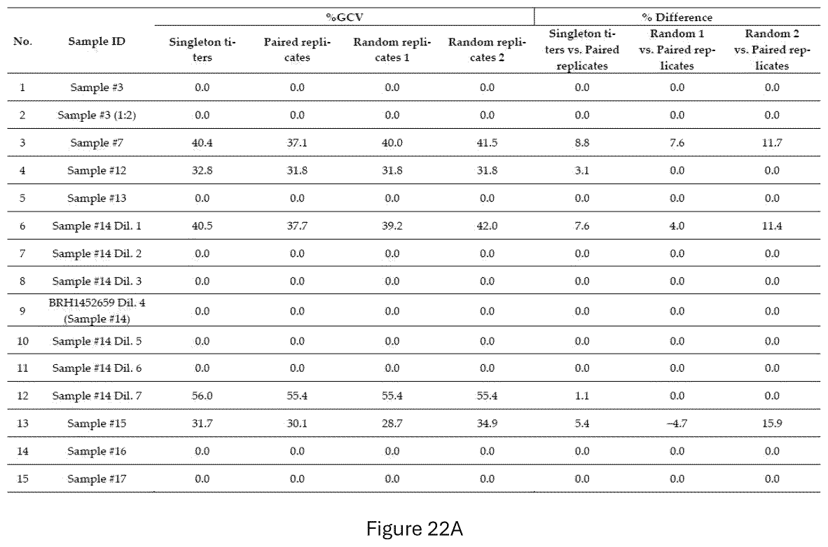

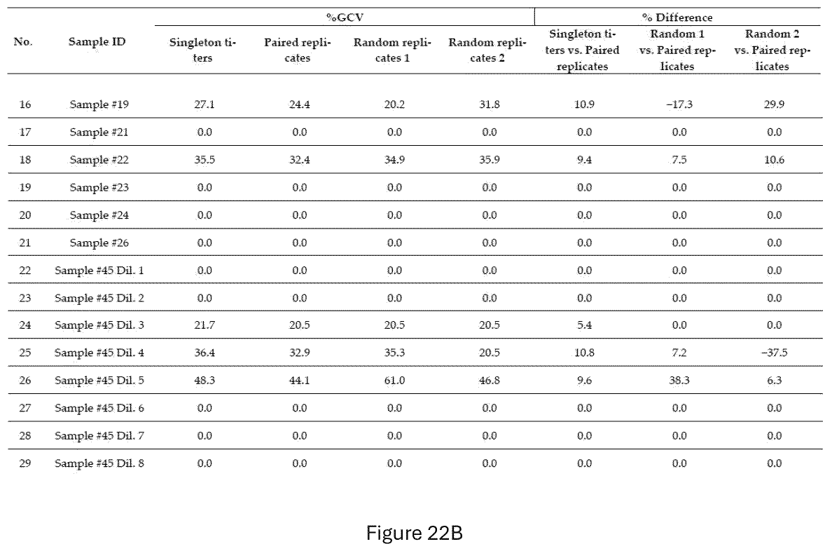

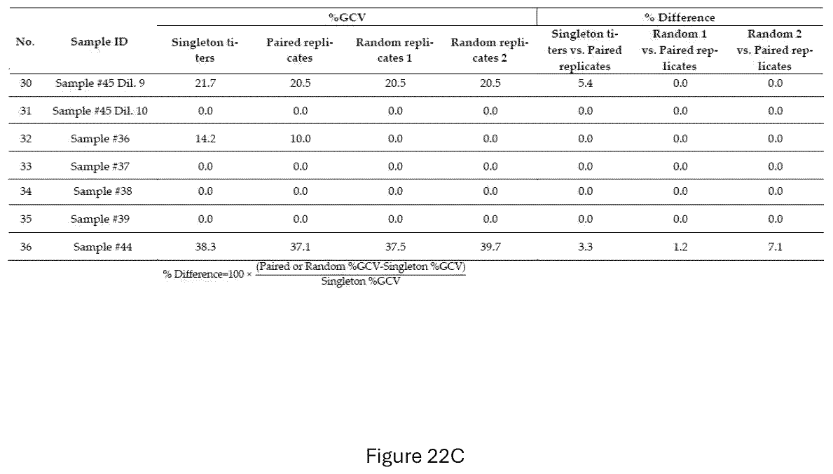

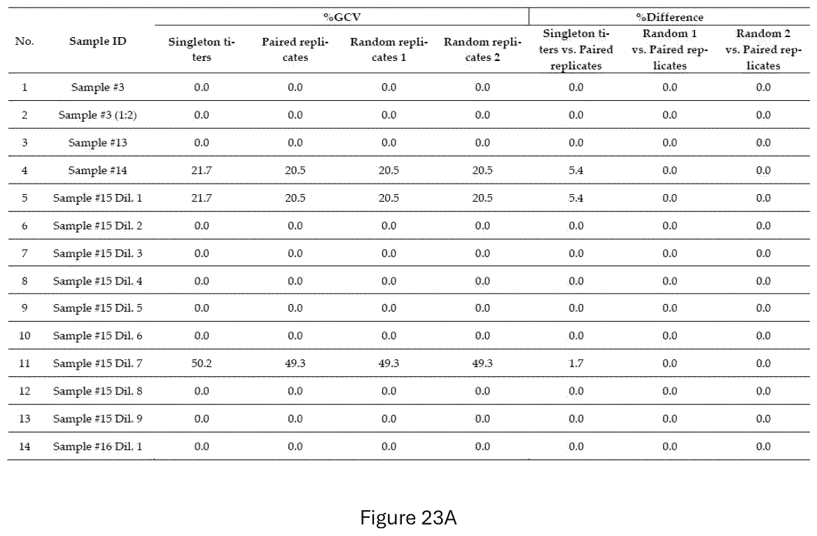

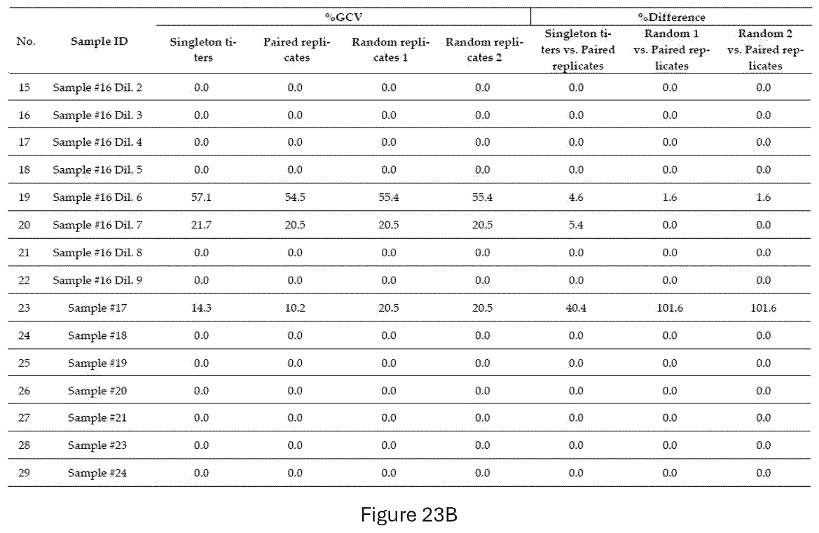

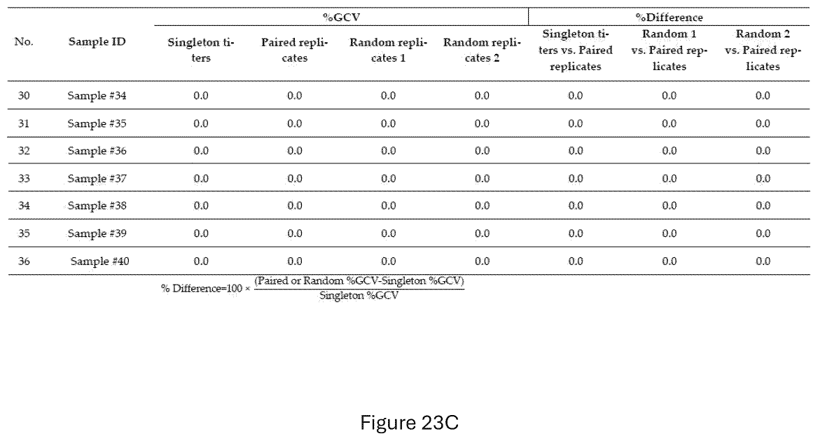

Inspection of the individual sample level results for all four vaccine-homologous strains (A/Kansas/14/2017, A/Brisbane/02/2018, B/Phuket/3073/2013, and B/Maryland/15/2016) suggests that the use of singleton titers, paired replicates, or random replicate data has little influence on precision, as can be seen in FIGS. 20A-20C, 21A-21C, 22A-22C, and 23A-23C. Overall precision for each vaccine-homologous strain was virtually unaffected by the various replicate strategies as assessed by % GCV at the inter-, intra-, and total-assay levels (FIG. 9).

### Influence of Singleton (Single Titer) Testing on Clinical Study GMT Results

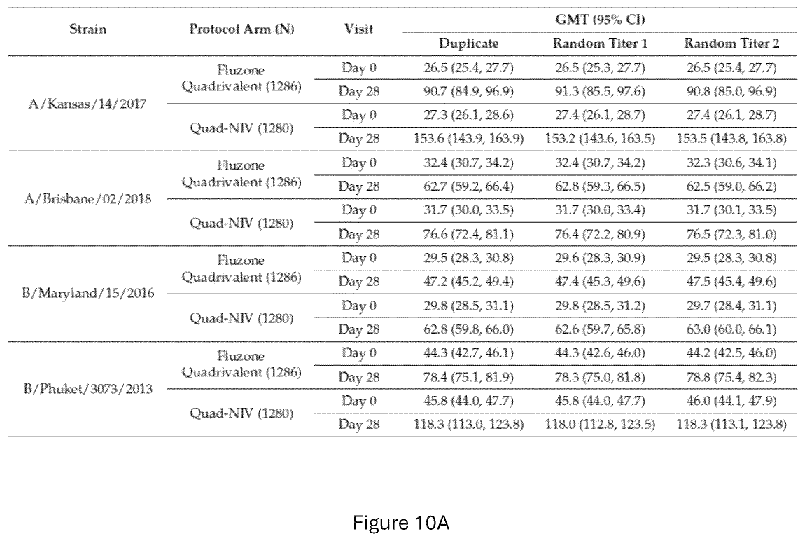

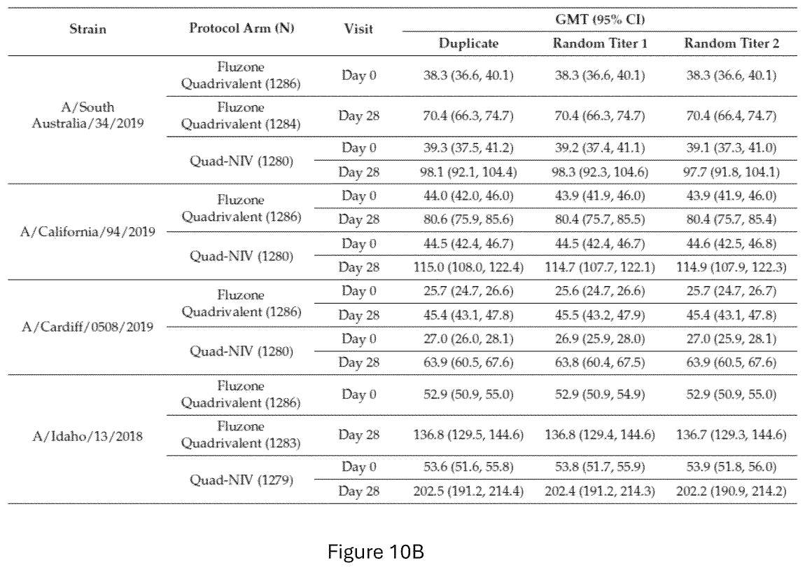

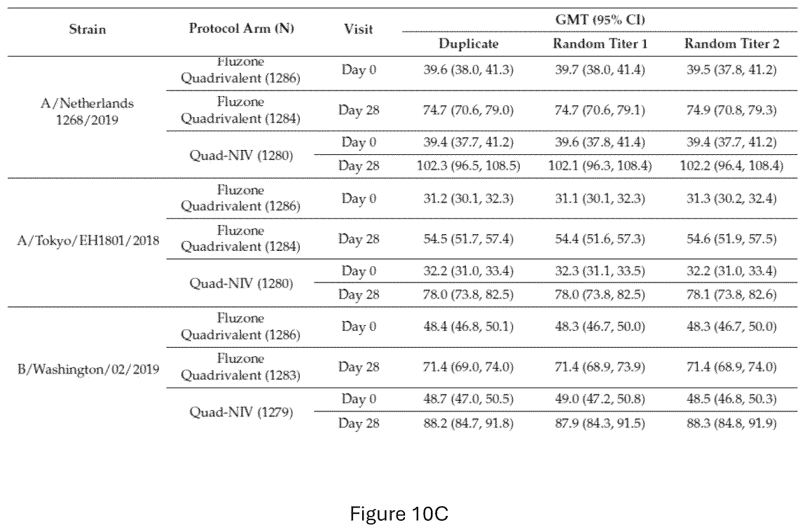

There was no noticeable difference in the GMT determined using the paired duplicate results or randomly selected titers for all the VLP strains and vaccine groups (FIGS. 10A-10C; such as for A/Kansas/14/2017: GMT [95% confidence interval {CI}] at Day 28 for 2019-2020 Fluzone Quadrivalent vaccine groups were 90.7 [84.9, 96.9], 91.3 [85.5, 97.6], and 90.8 [85.0, 96.9], respectively, for duplicates, random 1 and random 2). Since the GMTs were very close (no statistical difference) with an almost overlapping 95% CI between the singleton and duplicates, testing samples in singleton had no appreciable influence on clinical GMT results.

### Influence of Singleton (Single Titer) Testing on Clinical Study GMR, SPR, and SCR Results

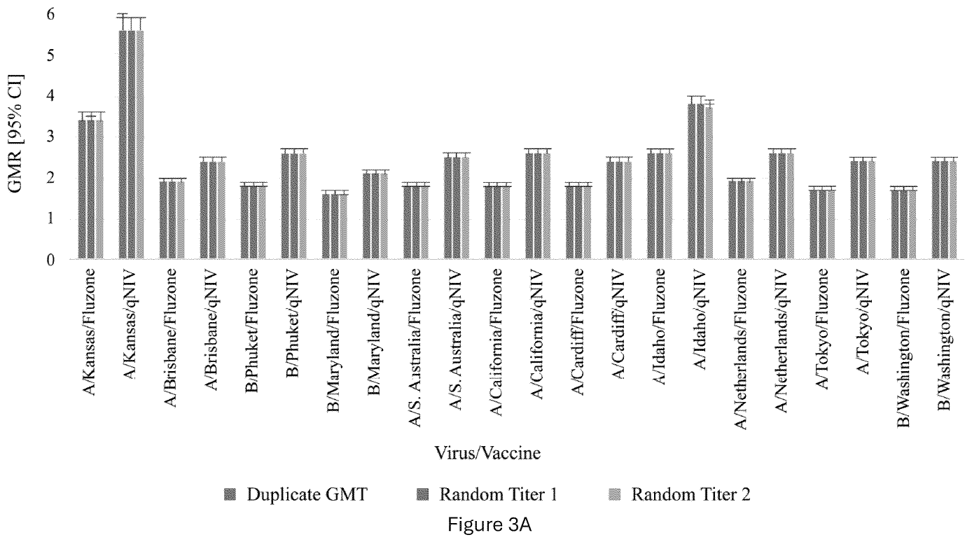

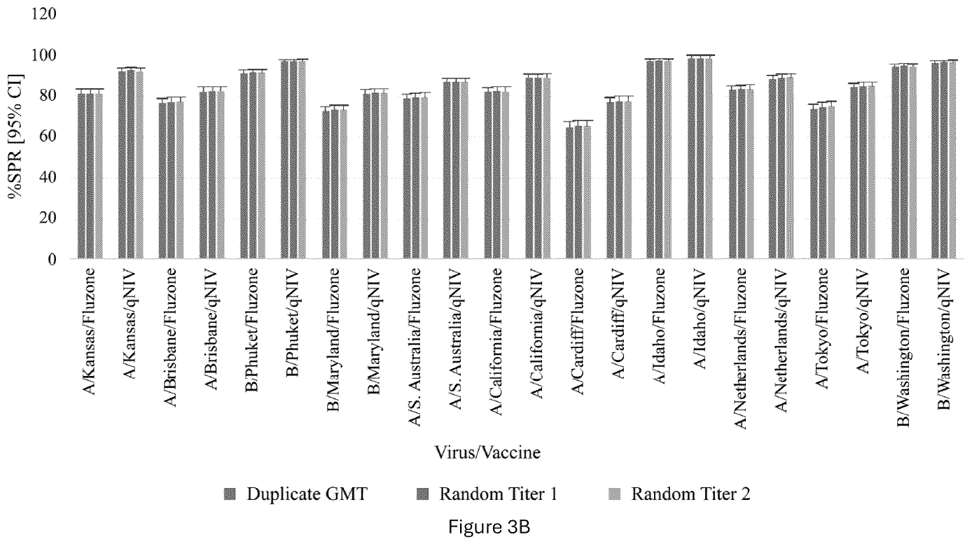

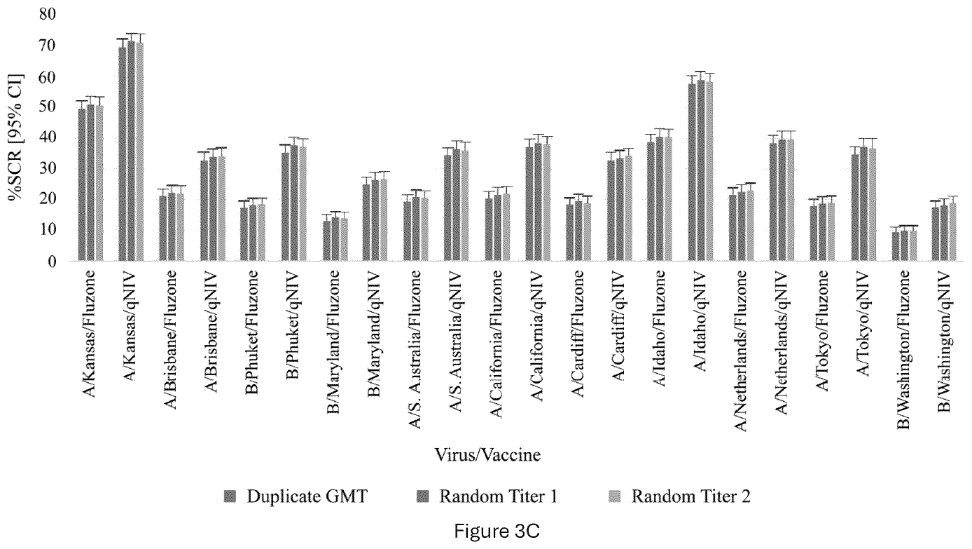

The GMR, % SPR, as well as % SCR, and their corresponding 95% CI from paired duplicates or randomly selected single titers were very similar for each virus strain and protocol arm pair (FIG. 3A-3C). Overall, these findings showed that reporting titers in singleton had no impact on the four key clinical metrics of GMT, GMR, SPR, and SCR.

### Correlation Between VLP-Based HAI and Wild-Type Virus MN Assays

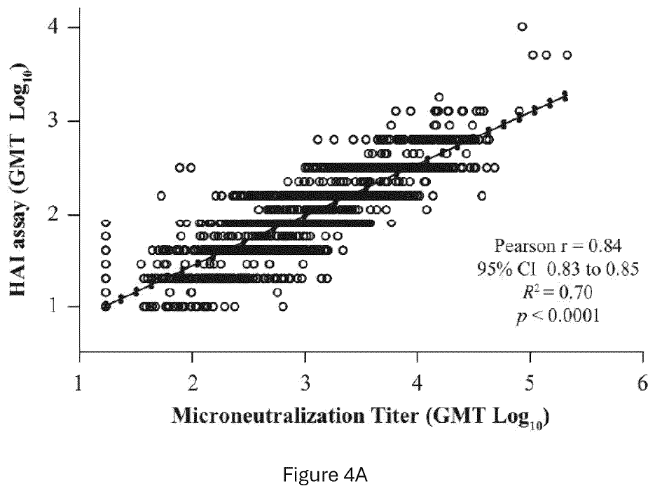

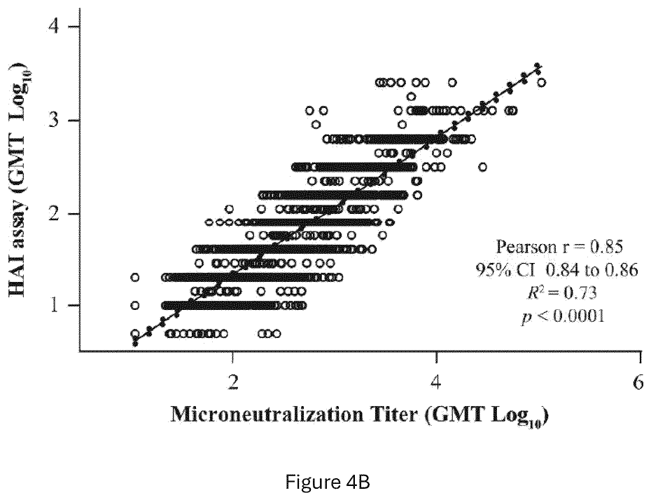

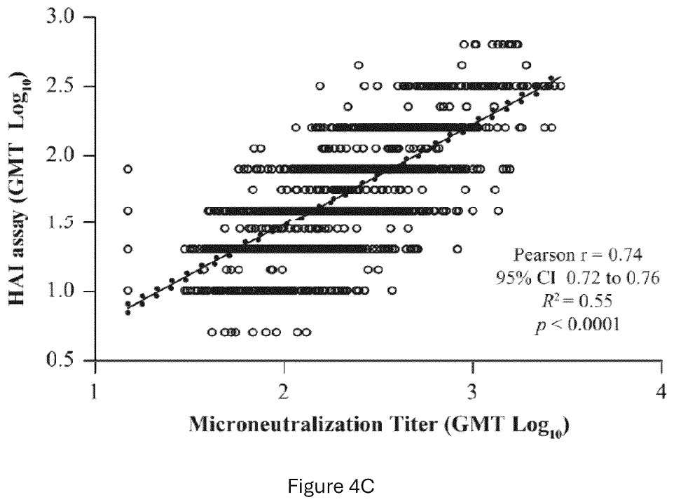

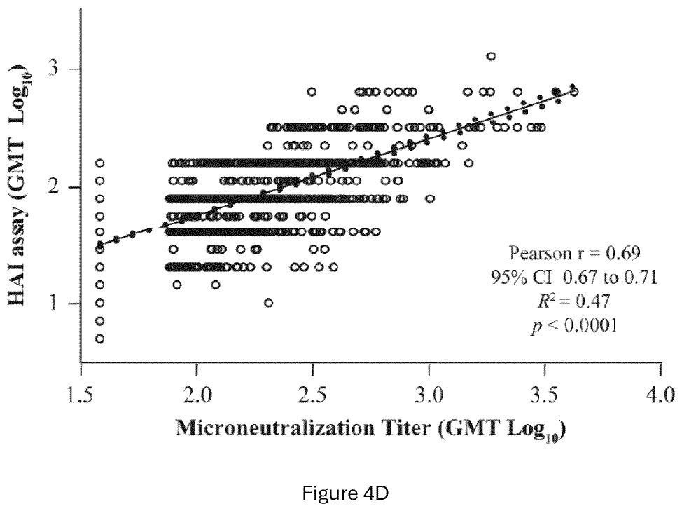

To evaluate the concordance of the HAI results with the MN assay, correlation analysis was performed using four different influenza strains. Results of the HAI for both A and B strains demonstrated a significant positive correlation with the qualified MN assays: A/Brisbane/02/2018 (Pearson's r=0.84, R2=0.70, p<0.0001; FIG. 4A), A/Kansas/14/2017 (Pearson's r=0.85, R2=0.73, p<0.0001; FIG. 4B), B/Maryland/15/2016 (Pearson's r=0.74, R2=0.55, p<0.0001; FIG. 4C), and B/Phuket/3073/2013 (Pearson's r=0.69, R2=0.47, p<0.0001; FIG. 4D).

### DISCUSSION

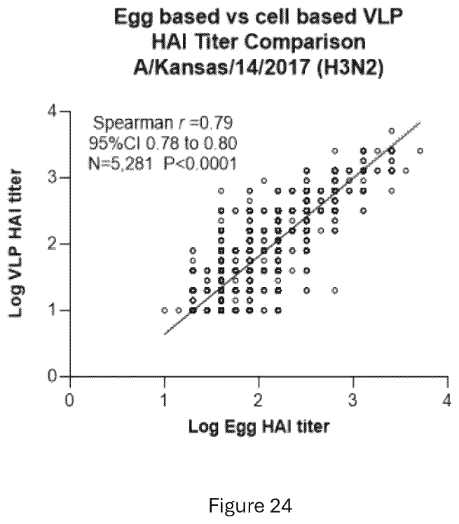

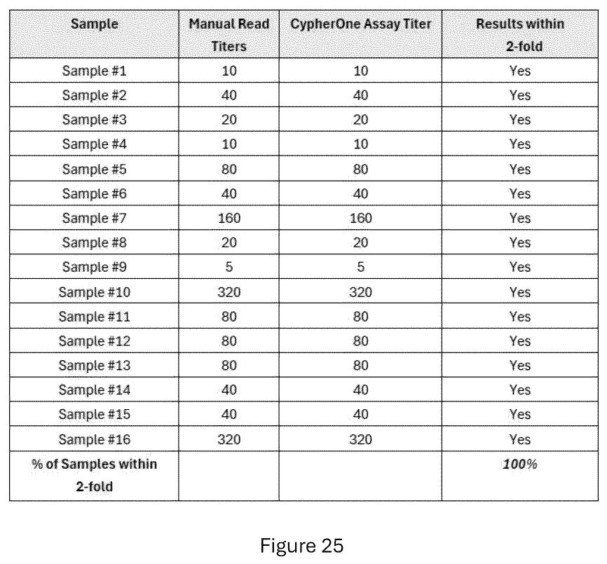

The data presented herein describes the development and validation of a novel robust HAI assay using egg-derived viruses/recombinant VLPs with human RBCs to assess the immunogenicity of seasonal influenza vaccines in clinical trial settings. To our knowledge, this is the first report that uniquely demonstrates the validation of HAI using human RBCs and an automated HAI reader suitable for clinical samples. The HAI assay showed acceptable precision, specificity, linearity, sensitivity, and robustness (RBC age [storage time], serum-virus/VLP incubation time, plate reading time) for the egg-derived viruses as well as the recombinant VLP strains. Furthermore, the singleton analyses using data from the VLP HAI validation did not significantly impact the GMT or precision (% GCV) at the individual sample level. Similarly, using the HAI titer data for wild-type sequence VLP from the Phase 3 clinical study qNIV-E-301 showed no adverse impact of reporting titers in singleton on all four key clinical metrics (GMT, GMR, SPR, and SCR). We have demonstrated that our novel modification of HAI is strongly predictive of influenza neutralizing activity in sera (FIG. 4A-4D) and have also explored the relationship between HAI titers using A (H3N2) VLPs and egg-derived viruses, which showed a strong positive correlation as well as a regression slope close to 1 (see, FIG. 24). We also note that the original historic driver of the adoption of HAI assays lay in their ability to predict neutralization data and protection (at the in vitro and animal challenge levels. We believe that the data presented in FIG. 4 of the present study strongly validate the HAI assay as fulfilling this function. We compared a CypherOne™-based readout and the manual reading of the assays using human RBCs during assay development (FIG. 25) and demonstrated equivalency through bridging experiments.

The source and the quality of the RBCs as critical reagents are reported as critical factors in the HAI assay. In general, RBCs of avian or animal origin are used in HAI assays. The selection of the appropriate species source of RBCs for the HAI assay remains a challenge given the failure of some recent H3N2 influenza viruses to agglutinate the RBC of common reagent species and non-specific inhibition of hemagglutination (because of non-specific serum interference with hemagglutination of avian RBCs). To circumvent these challenges, the present assay protocol used human RBCs (Type O) for the measurement of HAI antibody titers in human serum. Human influenza viruses preferentially bind to α2,6-linked sialic acid (SA) molecules. Human RBCs (Type O) and guinea pig RBCs have more α2,6-linked SA molecules on their surfaces compared with that of avian RBCs. Makkoch et al. (Erythrocyte Binding Preference of Human Pandemic Influenza Virus A and Its Effect on Antibody Response Detection. Ann. Lab. Med. 2012, 32, 276-282) showed that human RBCs (Type O) that lack preexisting antibodies against the pandemic influenza H1N1 virus yielded HAI titers most comparable to those obtained with turkey RBCs.

Additionally, the assay employed the NA inhibitor oseltamivir to prevent NA-mediated agglutination, which further strengthened the antigenic characterization of HA proteins of seasonal influenza viruses. Although the HAI GMT % difference (−50% to 100%) for both the egg-derived and VLP HAI assay results leveraged the use of RBC suspensions stored at 2 to 8° C. for up to 2 weeks, an asymmetric change/trend (−50% to 0%) in HAI GMT was noted for three strains in the VLP HAI assay. These findings recommend a maximum shelf-life of 1 week (7 days) as the most conservative condition for RBC suspension storage (2 to 8° C.) in the HAI assays, but surprisingly, results remain useful for RBC suspensions stored for longer periods (e.g., up to 2 weeks). Our study results are in line with a previous study that reported a shelf-life of up to 1 week for human erythrocytes. Additionally, it might be useful to prescreen RBCs from donors and virus combinations using QC samples before testing clinical trial sera to avoid any non-specific virus-RBC interaction.

The lack of harmonized and standardized assay readouts can introduce a high degree of variability in HAI antibody titer interpretation. The tilting of HAI plates at a 45±60° angle to check for a “teardrop pattern” in case of non-specific inhibition with avian RBCs is a commonly followed practice in many research laboratories. Similar to Wilson et al. (Automated Interpretation of Influenza Hemagglutination Inhibition (HAI) Assay: Is Plate Tilting Necessary? PLOS ONE 2017, 12, e0179939.), the present study used an automated platform CypherOne™ Hemagglutination Analyzer for reading HAI plates. The benefits of this platform are numerous, including the following: increased objectivity; standardization, consistency of titer interpretation; improved data integrity; reduced assay time, testing costs, possible data transcription errors; and the elimination of subjective biases in the manual scoring of plates as well as a permanent visual image/record to refer back to. Together, these factors indicate that the CypherOne™ platform adds significant value to clinical sample testing.

Validation of the HAI method ensures the reliability and reproducibility of results. Our study validated the egg-derived virus/recombinant VLP HAI assay for precision, specificity, linearity, sensitivity (LLOQ), RBC storage time, serum-virus/VLP incubation time, plate reading time, and serum sample storage and freeze/thaw effects on serum samples. A comprehensive understanding of these validation parameters is crucial for the assessment of clinical samples and assay reliability. In our study, the two HAI methods using human RBCs and either egg-derived influenza viruses or recombinant VLPs met the precision and accuracy criteria in measuring antibody titers in human serum, with overall assay % GCV being <30% for all egg-derived virus/VLP strains, and % relative bias within the range of −50% to 100% (2-fold). Neat (undiluted) sample stability findings showed that serum samples could be used in the HAI assays after up to seven freeze/thaw cycles and after 1 month of storage in a −20° C. freezer. These observations indicate that the validated HAI assays can be used in clinical trials to assess the immunogenicity of egg-derived and recombinant influenza vaccines.

Furthermore, testing samples in singleton in HAI assays can offer advantages in terms of reduced clinical testing time and costs, increased sample throughput, lower resource needs, and improved time management. Similarly, utilizing the HAI titer data of wild-type sequence VLP HAI from the Phase 3 clinical study and reporting titers from singleton showed no influence on the four key clinical analysis metrics such as GMT, GMR, SPR, and SCR. This suggests that the seasonal influenza VLP HAI assay samples (pre- and post-vaccination) can be tested routinely in singleton, and the final results can be reported as titers without adversely affecting the data readout and interpretation.

The HAI assay is a more commonly used serological technique for assessing influenza-specific humoral immunity than the MN assay. The HAI assay identifies antibodies that bind to the globular head of viral HA and prevent virus-mediated RBC agglutination, while the MN assay detects functional neutralizing antibodies inhibiting the virus entry/replication in mammalian cells, which may have a broader range of specificities. Given these differences, establishing correlations between the HAI and MN assays would be valuable for comparing vaccine assessments and gaining a comprehensive understanding of immune responses to influenza vaccines. Our study results revealed a significant correlation between the validated HAI assay and a qualified MN assay (for both A and B strains). These findings indicate that the validated HAI assay results align with other measures of the immune response against seasonal influenza. Strong correlation observed between HAI and MN results in our studies are consistent with previous publications (Veguilla et al. (Sensitivity and Specificity of Serologic Assays for Detection of Human Infection with 2009 Pandemic H1N1 Virus in U.S. Populations. J. Clin. Microbiol. 2011, 49, 2210-2215.) and Trombetta et al. (Comparison of Hemagglutination Inhibition, Single Radial Hemolysis, Virus Neutralization Assays, and ELISA to Detect Antibody Levels against Seasonal Influenza Viruses. Influenza Other Respir. Viruses 2018, 12, 675-686.)), which also showed strong positive correlations between the HAI and MN assays for both the A and B influenza strains.

### CONCLUSIONS

In conclusion, the validated egg-derived virus and VLP HAI assays with human RBCs performed similarly with comparable precision and accuracy, indicating the suitability for evaluating humoral immune response (HAI) for seasonal influenza in human serum samples from clinical studies. Furthermore, there was no difference in the precision, GMR, SPR, and SCR with singleton determination of titer testing compared with duplicate titer HAI testing. These findings suggest that testing in singleton can offer cost-effectiveness and improve assay throughput, without impacting key study parameters and conclusions from clinical data analysis.

1. A method for determining if a biological sample contains antibodies that inhibit influenza virus hemagglutinin (HA) glycoprotein hemagglutination activity, comprising:
a. reducing or removing non-specific inhibitors of HA from the biological sample to produce a treated sample;
b. exposing the treated sample to an influenza virus having an HA glycoprotein to produce a sample-virus mixture;
c. incubating the sample-virus mixture with human red blood cells (RBCs), the human RBCs being at a concentration of about 0.5% to about 1%; and
d. detecting agglutination of the human RBCs;
wherein the biological sample contains antibodies that inhibit HA glycoprotein hemagglutination activity if agglutination of the RBCs is not detected.

2. The method of claim 1, wherein the biological sample is serially diluted, and each serial dilution of the sample is subjected to steps a, b, c, and d.

3. The method of claim 2, comprising determining the hemagglutination inhibition titer of the biological sample.

4. The method of claim 1, wherein the human RBCs are at a concentration of about 0.75%.

5. The method of claim 1, wherein the sample-virus mixture is incubated with the human RBCs for about 60 minutes to about 150 minutes, or about 80 minutes to about 100 minutes.

6. The method of claim 1, wherein step a is performed using a receptor-destroying enzyme (RDE).

7. The method of claim 1, wherein the sample-virus mixture includes 2 to 10 hemagglutination (HAg) units, or 3 to 5 HAg units, or 4 HAg units of virus.

8. The method of claim 1, wherein the virus comprises an egg-derived influenza virus.

9. The method of claim 1, wherein the virus comprises a virus-like particle (VLP).

10. The method of claim 1, wherein the virus is treated with oseltamivir.

11. The method of claim 1, wherein step d is performed using an automated image reader-based analysis of hemagglutination.

12. The method of claim 1, wherein the method is performed once per biological sample.

13. A kit for use in a method of claim 1, comprising:
a. an influenza virus having an HA glycoprotein; and
b. human RBCs or an RDE.

14. The kit of claim 13, wherein the virus comprises an egg-derived influenza virus.

15. The kit of claim 13, wherein the virus comprises a VLP.

16. The kit of claim 13, wherein the kit comprises oseltamivir or the influenza virus has been treated with oseltamivir.

17. The kit of claim 13, comprising at least one of:
a. a dilution buffer;
b. a multi-well assay plate; and
c. an RDE.

18. The kit of claim 17, wherein the dilution buffer comprises Dulbecco's phosphate buffered saline.

19. A method for selecting an immunogenic composition for use as a vaccine, comprising:
a. obtaining a plurality of first biological samples from subjects that have been administered a first immunogenic composition directed to a seasonal influenza virus;
b. performing a method according to claim 4 using the plurality of first biological samples to determine the hemagglutination inhibition titers of the first biological samples; and
c. selecting the first immunogenic composition for use as a vaccine if the hemagglutination inhibition titers in the plurality of first biological samples meets or exceeds a predetermined threshold.

20. The method of claim 19, comprising:
d. obtaining a plurality of second biological samples from subjects that have been administered a second immunogenic composition directed to a seasonal influenza virus;
e. performing a method according to claim 4 using the plurality of second biological samples to determine the hemagglutination inhibition titers of the second biological samples;
f. comparing the hemagglutination inhibition titers of the plurality of first biological samples to the hemagglutination inhibition titers of the second biological samples; and
g. selecting the immunogenic composition that was administered to the subjects with biological samples having higher hemagglutination inhibition titers for use as a vaccine.
